(19) United States
(12) Patent Application Publication
(10) Pub. No.: US 2019/0313774 A1
(43) Pub. Date: Oct. 17, 2019

(54) Tissue Attachment Device And Method

(71) Applicant: Zift Medical, LLC, Excelsior, MN (US)

(72) Inventors: Robert S. Schwartz, Inver Grove Heights, MN (US); Stanton J. Rowe, Newport Coast, CA (US); Robert A. Van Tassel, Excelsior, MN (US); Ralph Schneider, Trabuco Canyon, CA (US); Ming Wu, Tustin, CA (US); David John Blaeser, Brooklyn Park, MN (US); Philip Jon Haarstad, Chanhassen, MN (US); Eric J. Simso, Minnetrista, MN (US); Douglas Jay Krone, Rogers, MN (US); Brian Zelickson, Minneapolis, MN (US); Robert A. Ganz, Minnetonka, MN (US); Matt Blaeser, Brooklyn Park, MN (US); Matt Keillor, Inver Grove Heights, MN (US)

(73) Assignee: Zift Medical, LLC, Excelsior, MN (US)

(21) Appl. No.: 16/453,956

(22) Filed: Jun. 26, 2019

Related U.S. Application Data
(63) Continuation-in-part of application No. 15/858,091, filed on Dec. 29, 2017.
Application No. 15/858,091 is a continuation-in-part of application No. 14/941,429, filed on Nov. 13, 2015.
Application No. 14/941,429 is a division of application No. 13/961,785, filed on Aug. 7, 2013, now abandoned.
(60) Provisional application No. 61/680,663, filed on Aug. 7, 2012.

(51) Int. Cl. A45D 44/22; A61B 17/064; A61B 17/068; A61B 17/84

(52) U.S. Cl. CPC A45D 44/22; A61B 17/064; A61B 17/068; A61B 2017/00986; A61B 17/846; A61B 17/84; A61B 17/0642; A61B 17/0682

(57) ABSTRACT

A method and apparatus for attaching tissue to bone in a shifted position without requiring surgical detachment of muscle or connective tissue joining the tissue layer to the bone layer. The skin layer is gently pulled in a non-surgical manner and a fastener of the invention is driven through the skin layer into the bone layer to effect a “skin tightening” procedure.

### RELATED APPLICATIONS

This application is a Continuation-In-Part of and claims priority to U.S. patent application Ser. No. 15/858,091 filed Dec. 29, 2017 entitled Tissue Attachment Device and Method, which is hereby incorporated herein by reference in its entirety; and is also a Continuation-In-Part of and claims priority to U.S. patent application Ser. No. 14/941,429 filed Nov. 13, 2015 entitled Tissue Attachment Device and Method, which is a divisional application of and claims priority to U.S. patent application Ser. No. 13/961,785 filed Aug. 7, 2013 entitled Tissue Attachment Device And Method (now abandoned), which claims priority to U.S. Provisional Application Ser. No. 61/680,663 filed Aug. 7, 2012 entitled Tissue Attachment Device And Method, all of which are hereby incorporated herein by reference in their entireties.

### FIELD OF THE INVENTION

This invention generally relates to a method and apparatus for tissue attachment, with one or more tissue layers being attached to bone or other tissue layers. There are multiple applications, including but not limited to dermatology/plastic surgery among others. The application specifically described in this disclosure is to reposition the skin and/or surrounding tissues, of the face or any other body part in relationship to associated bone, cartilage or tissue structures, then secure the tissue in the new position. The overall effect is to restore tissue to a preferred position, such as might be done in a brow-lift or other cosmetic procedure or in orthopedic procedures such as tendon reattachment.

### BACKGROUND OF THE INVENTION

The current standard facelift procedure (technically known as a rhytidectomy) involves the surgical removal of excess skin and tissue from the face and the redraping of the remaining skin on the face and neck. The procedure involves making an incision in front of the ear, which extends up into the hairline and wraps around the bottom of the ear and behind it, ending in the hairline on the back of the neck. After the incision is made, the skin and/or underlying tissues are separated from the bone and or periosteum using blunt dissection, t, and the skin redraped and sutured in the new position, with the excess skin being removed. A variation of this procedure is the endoscopic facelift in which a series of smaller incisions (10-30 mm) are made at or above the hairline, and using blunt dissection and endoscopic guidance the tissue is released from the underlying bone. A variety of techniques such as sutures, screws, bone anchors, bone tunnels and various implants are used to elevate the tissue and secure the tissue to the underlying bone. An even more limited approach is the Brow-pexy in which small (1-2 cm) incisions are made below the brow, and blunt dissection is again used to separate the soft tissue from underlying bone and various means are used to secure the soft tissue in an elevated position.

All variations of aesthetic procedures are expensive, involve a long and painful recovery period, and may be accompanied by complications such as infection, bleeding, nerve damage and complications from anesthesia.

Efforts have been directed lately toward various techniques that involve fixation of skin through the use of permanent or resorbable tacks or anchors to effect facial rejuvenation. However, these procedures still involve the use of significant incisions, requiring a substantial recovery period, and associated surgical, anesthesia, and post-surgical risks. There is thus a need in dermatology and plastic surgery for a less invasive, less expensive method for reducing the effects of ageing and restoring tissue to it's natural position on the face and other areas of the body. Such a method would also have utility in the treatment of facial paralysis due to stroke, Bells Palsy, or surgical or other trauma. The procedure might also have utility in treating patients with traumatic injuries to the face such as might be caused by automobile accidents or battlefield injuries. The procedure might also have utility in treating common tendon avulsion injuries such as mallet or baseball finger.

Methods and devices that addresses the above need has been developed and are shown and described in U.S. application Ser. No. 13/961,785 filed Aug. 7, 2013 entitled Tissue Attachment Device And Method, incorporated by reference herein in its entirety. The methods and devices are generally directed to accomplishing the steps of moving the soft tissue layer to a new position in relation to the bone or cartilage below; having a device which is either pushed or ballistically and dynamically driven into the deep layer of bone, cartilage or soft tissue through a superficial soft tissue layer, thereby holding the superficial soft tissue layer in the new position in relation to the bone, cartilage or soft tissue through the use of anchors.

Prior to the development of this technology, little or no experimentation had been performed in the area of ballistically firing micro-pins into the tissue/bone layers of the human skull. Since the filing of the aforementioned application, much has been learned about the dynamics of firing these micro-pins into a variety of bones, resulting in variations in methods and designs in both the pins and delivery devices. At least some of these variations are described herein.

### SUMMARY OF THE INVENTION

Several embodiments are shown and described herein directed to percutaneously lifting, translating, and ‘tightening’ the skin, effecting a reduction in the appearance of wrinkles and excess skin on the face and other areas of the body. The method of the present invention enables a minimally invasive procedure for correcting aging of the face through “skin tightening.” The methods and devices are generally directed to accomplishing the steps of moving the soft tissue layer to a new position in relation to the bone or cartilage below; having a device which is either pushed or ballistically and dynamically driven into the deep layer through a superficial soft tissue layer, thereby holding the soft tissue layer in the new position in relation to the bone or cartilage through the use of anchors.

For example, the methods and devices of the invention are useful in facial procedures, such as effecting a reduction in the appearance of wrinkles and excess skin on the face and other areas of the body an/or the translation of the skin and/or underlying tissues in order to correct the position of the skin and associated anatomical features. In one application, the device could be used to reposition the brow to help correct for brow ptosis and/or visual field impairment. Another example would be to correct for facial asymmetry caused by muscle paralysis due to stroke or Bell's palsy. Another example would be to correct facial deformities due to trauma of various kinds. The method of the present invention enables a minimally invasive procedure for repositioning the skin and associated anatomical features.

The methods and devices of the invention are useful in other, non-facial applications as well. For example, the devices and methods are well-suited for reattaching tendons, such as in the hand or other areas.

More generally, one aspect of the invention provides a method for relocating a skin and/or underlying tissues layer relative to an adjacent bone layer comprising: shifting a skin and/or underlying tissues layer relative to an adjacent bone layer from an original position to a shifted position without cutting deep muscle or connective tissue associated with the skin layer; holding the skin layer and underlying tissues in the shifted position while driving a fastener through the skin layer into the bone layer a desired depth to prevent the skin layer and/or underlying tissues from reassuming the original position; wherein driving the fastener through the skin layer into the bone layer comprises transferring a minimum amount of energy to the fastener.

Transferring a minimum amount of energy to the fastener may comprise accelerating the fastener to a minimum velocity. The minimum velocity may be determined by a size of the fastener. Alternatively, the minimum velocity may be determined by a size of a predrilled hole in the bone relative to a size of the fastener. The size of the predrilled hole may comprise a depth of the predrilled hole. The size of the predrilled hole may comprise a width of the predrilled hole.

Transferring a minimum amount of energy to the fastener may be accomplished by accelerating the fastener with a firing pin.

The depth the fastener is driven into the bone layer may be controlled by a travel length of the firing pin or by applying a control level of energy to the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end and a proximal end, the hollow shaft including: an anchoring feature proximate the distal end; and, a tissue-holding feature proximate the proximal end; and a spike housed within the hollow shaft and protruding from a distal end thereof, the spike including a sharpened tip.

In one aspect of the invention, the method includes the introduction of a substance, for example an adhesive (e.g. fibrin glue) to bond the device to the bone or bond the tissue to the bone. Additionally or alternatively, the substance could be one that promotes healing. The substance could be introduced via the delivery device or through the implant itself, either through the implant or applied to the implant as a coating.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end that includes a sharpened leading edge and a proximal end, the hollow shaft including: an anchoring feature proximate the distal end; and, a tissue-holding feature proximate the proximal end.

The tissue-holding feature may comprise at least one petal that flares outwardly upon deployment. At least one petal may comprise a memory metal.

The anchoring feature may comprise at least one barb that flares outwardly upon deployment.

The anchoring feature may comprise at least one petal that flares outwardly upon deployment

The distal end of the hollow shaft may comprise a bevel that aligns with a surface of the sharpened tip.

The sharpened tip may comprise a flange that has a diameter greater than a diameter of the hollow shaft.

In some applications, the tissue-holding ability of the device may be enhanced by forming a dissection plane in the targeted tissue. The dissection plane is formed by releasing underlying tissue to induce scarring, thereby taking advantage of the increased durability that scar tissue provides. We have contemplated releasing the underlying tissues sub-periosteally or super-periosteally through the same or a different small incision prior to placement of anchors. This can be accomplished utilizing a small tool, such as a blunt dissecting tool or elevator, that would be placed through the small incision.

Implantation can be accomplished super-periosteally.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener having a hollow shaft with a distal end and a proximal end, the hollow shaft including: a tissue-holding feature proximate the proximal end; and a screw housed within the hollow shaft and protruding from a distal end thereof.

The tip of the screw may comprise as self tapping feature at its distal end. Alternatively or additionally, a hole may be pre-drilled in the bone. It is likely that the size of the screw for a given application may dictate whether pre-drilling is merited.

Another aspect of the invention provides an implant for use in anchoring a skin layer and/or underlying tissues in a shifted position to a bone layer thereby overcoming lateral and axial forces imparted by the skin layer and underlying tissues on the implant comprising: a fastener having a screw like element with a proximal and distal end. The proximal end may have a section of increased diameter to interact with a tissue holding feature. A tissue holding feature that is configured to accept a screw like feature.

The tissue holding feature may be made from a flat sheet or hollow tube.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to the bone and a tissue holding feature that is displaced laterally some distance away from the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to periosteal tissue and a tissue holding feature.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a fastener fixed to periosteal tissue and a tissue holding feature that is displaced laterally some distance away from the fastener.

Another aspect of the invention provides an implant for use in anchoring a skin layer and/or underlying tissues in a shifted position to a bone layer thereby overcoming lateral and axial forces imparted by the skin layer and/or underlying tissues on the implant comprising: a tissue holding feature that is displaced laterally some distance away from another tissue holding feature.

Another aspect of the invention provides an implant for use in anchoring a skin and/or soft tissue layer that includes a tissue holding feature that is adjustably displaced from an anchor and/or from another tissue holding feature. In addition to being adjustable, one embodiment provides a connecting element between the tissue holding feature and the fastener (or another tissue holding feature) that expands and contracts longitudinally. Doing so allows for tissue relaxation over time.

Another aspect of the invention provides a device for use in anchoring a skin layer and/or underlying tissues in a shifted position relative to a bone layer comprising: a firing gun generally including an energy source, a firing pin and an implant.

One embodiment of the device includes a handle; a firing mechanism disposed within the handle and including a firing pin and a trigger mechanism; a disposable tip removably attached to a distal end of the firing gun and including: a proximal tip component that removably attaches the disposable tip to the distal end of the firing gun; a distal tip component distally attached to the proximal tip component; an implant carried within the distal tip component; wherein when the trigger mechanism is pulled: the firing mechanism is activated, thereby transferring energy to the firing pin; the firing pin in turn accelerates the implant to at least a minimum velocity, thereby driving the implant into an implantation site.

The firing mechanism may comprise a spring.

The firing mechanism may comprise a compressed gas.

The firing mechanism may comprise an electronic solenoid or similar electromechanical feature.

The firing mechanism may comprise a hydraulic fluid.

The proximal tip component may slidingly attach to the distal tip component.

The safety feature may require the firing gun to be compressed against a surface in order to activate the triggering mechanism.

Another aspect of the invention provides a fastener for use in holding a tissue layer in a shifted position relative to an adjacent tissue layer. The fastener includes a tissue holder for holding the shifted layer and an anchor for fixing the fastener relative to the adjacent tissue layer, such as the periosteum or bone layer. The tissue holder is connected to the anchor with a shaft that runs substantially parallel to the adjacent tissue layer. The lateral displacement between the tissue holder and the anchor or anchors may be variable. Additionally, there may be one or more tissue holders positioned either adjacent to each other, or laterally displaced from each other. The tissue holders may be round, oblong or angled.

The anchor may include one or more barb or other fasteners such as spikes, screws or tacks. The anchors may be designed to engage any soft tissue, such as the periosteum, or the bone layer.

The fastener may be constructed of a memory metal, such as nitinol, such that the tissue holder may transform from a straight, delivered configuration, to a curled deployed configuration upon release from a delivery device. The tissue holder, anchor, or both may be constructed from a biodegradable/bioabsorbable material.

The delivery device may be a low-profile catheter or cannula from which the fastener is ejected during delivery. The fastener may be loaded into the delivery device such that the anchor side of the fastener is distal and emerges first, or it may be loaded such that the tissue holder is distal and emerges first.

One embodiment of the method of the invention involves using a delivery device that causes anchors to be impelled at high velocities into the deep layer such as bone whereby they are stopped after a certain distance by the hard layer and become firmly lodged in that layer. The delivery device may be a handheld resembling a pen that has energy capability to accelerate the anchor into the deeper tissues.

In another embodiment, the delivery device comprises an adhesive strip with anchors already lodged in the strip and spaced apart from one another. A practitioner may measure the length of adhesive strip needed, apply it directly to desired area of patient's skin, and accelerate the anchors into the tissue individually.

One aspect of the invention provides a method for relocating a skin layer relative to an adjacent bone layer comprising shifting a skin layer relative to an adjacent bone layer from an original position to a shifted position without cutting muscle or connective tissue associated with the skin layer; holding the skin layer in the shifted position while inserting a fastener through the skin layer into the bone layer to prevent the skin layer from reassuming the original position. One or more additional fasteners may be subsequently inserted through the skin layer into the bone layer.

One aspect of the invention includes inserting a fastener through the skin layer into the bone layer comprises inserting a fastener through the skin layer into the bone layer such that an outer-most feature of the fastener is located just below an outer surface of the skin layer.

Another aspect of the invention involves inserting a fastener through the skin layer comprises ballistically inserting a fastener through the skin layer. This may be accomplished by driving the fastener into the bone layer using a spring-loaded driving mechanism. Alternatively, driving the fastener into the bone layer may be done by using a compressed-gas driving mechanism.

One or more embodiments may include a fastener with an open hollow tip. The fastener may also have an anchoring feature proximate a distal end thereof for preventing the fastener from becoming dislodged from the bone layer.

The invention also provides a fastener for use in anchoring a skin layer in a shifted position to a bone layer thereby overcoming lateral and axial forces imparted by the skin layer on the fastener comprising at least one shaft having a distal end and a proximal end, the at least one shaft including: an anchoring feature proximate the distal end; and, a tissue-holding feature proximate the proximal end.

In one aspect, the fastener comprises a plurality of shafts and the tissue holding feature comprises at least one bridge connecting one of the plurality of shafts to another one of the plurality of shafts.

In another aspect, the aforementioned at least one shaft comprises a hollow portion that is open at the distal end.

In another aspect, the anchoring feature comprises at least one barb.

In another aspect, the fastener comprises a bioabsorbable material.

In another aspect, the tissue holding feature comprises at least one protrusion that extends radially from a longitudinal axis of the shaft when the fastener is driven into the bone layer.

In another aspect the fastener includes a stop that limits a depth that the fastener can be driven into the bone layer.

The shaft of one or more of the fasteners of the invention may have a shaft that comprises a hollow portion that is open at the distal end and the stop comprises a closed proximal end of the hollow portion.

The invention also includes device for use in anchoring a skin layer in a shifted position to a bone layer thereby overcoming lateral and axial forces imparted by the skin layer on the fastener comprising at least one shaft having a distal end and a proximal end comprising: a delivery mechanism; and, at least one fastener; the delivery mechanism including: a barrel; a driving mechanism that drives the at least one mechanism out of a distal end of the barrel; the at least one fastener including at least one shaft having: an anchoring feature proximate the distal end; and, a tissue holding feature proximate the proximal end.

The delivery mechanism may further include a cartridge containing a plurality of the fasteners.

The driving mechanism may comprise a spring; a piston driven by the spring and having a distal end that transfers an axial force released by the spring onto a proximal end of the fastener; a triggering mechanism for releasing energy stored in the spring.

The driving mechanism may also comprise: a capsule containing pressurized gas; a piston driven by the pressurized gas and having a distal end that transfers an axial force onto a proximal end of the fastener; and a triggering mechanism for releasing the axial force stored in the spring.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIGS. 1A, 1A and 1C show the manner in which tissue is relocated using the present invention;

FIG. 2 is a cutaway view of an fastener of the present invention embedded into tissue;

FIGS. 3A and 3B show a patients face before and after wrinkles have been removed using the present invention;

FIG. 4 is an elevation of an embodiment of an fastener of the present invention;

FIG. 5 is an elevation of an embodiment of an fastener of the present invention;

FIG. 6 is a perspective view of an embodiment of an fastener of the present invention;

FIG. 7 is an elevation of an embodiment of an fastener of the present invention;

FIGS. 8A and 8B are elevations of an embodiment of an fastener of the present invention;

FIG. 9 shows an embodiment an fastener of the present invention being driven into bone;

FIG. 10 is a perspective view of an embodiment of a fastener of the present invention.

FIG. 11A is a perspective view of an embodiment of a fastener of the present invention.

FIG. 11A is a transparent perspective view of the embodiment of a fastener of the present invention shown in FIG. 11A.

FIG. 12 is a perspective view of an embodiment of a fastener of the present invention.

FIG. 13 is a perspective view of an embodiment of a fastener of the present invention.

FIG. 14 is a perspective view of an embodiment of a fastener of the present invention.

FIG. 15 is a perspective view of an embodiment of a fastener of the present invention.

FIG. 16 is a perspective view of an embodiment of a fastener of the present invention.

FIG. 17 is a perspective view of an embodiment of a fastener of the present invention.

FIG. 18 is a perspective view of an embodiment of a fastener of the present invention.

FIG. 19 is a perspective view of an embodiment of a fastener of the present invention.

FIG. 20 is a perspective view of the embodiment of a fastener of FIG. 19 loaded into a distal end of a delivery device.

FIG. 21 is a perspective view of an embodiment of a delivery device of the present invention;

FIG. 22 is a perspective view of an embodiment of a delivery device of the present invention;

FIG. 23 is a perspective view of an embodiment of a delivery device of the present invention;

FIG. 24 is a perspective view of an embodiment of a delivery device of the present invention;

FIG. 25 is a perspective view of an embodiment of an implant of the invention;

FIG. 26 is a perspective view of an embodiment of a spike of the invention;

FIG. 27 is a perspective view of an embodiment of an implant of the invention;

FIG. 28 is a perspective view of an embodiment of a spike of the invention;

FIG. 29A is a perspective view of an embodiment of an implant of the invention in a delivery configuration;

FIG. 29B is a perspective view of the embodiment of FIG. 7a in a deployed configuration;

FIG. 30 is a depiction of a step of an implantation procedure of the invention;

FIG. 31 is a depiction of a step of an implantation procedure of the invention;

FIG. 32 is a depiction of a step of an implantation procedure of the invention;

FIG. 33 is a depiction of a step of an implantation procedure of another embodiment of the invention;

FIG. 34 is an elevation of an embodiment of a fastener of the invention;

FIG. 35 is a perspective view of the fastener of FIG. 34 implanted on a patient;

FIG. 36 is a close-up view of the depiction of FIG. 35;

FIG. 37 is a depiction of an embodiment of a fastener of the invention and its position in relation to skin layers when implanted;

FIG. 38 is an elevation of an anchoring end of an embodiment of a fastener of the invention;

FIG. 39 is a perspective view of an embodiment of a fastener of the invention;

FIG. 40 is an elevation of an embodiment of a fastener of the invention;

FIG. 41 is an elevation of an embodiment of a fastener of the invention;

FIG. 42 is an elevation of an embodiment of a fastener of the invention;

FIG. 43 is an elevation of an embodiment of an anchor of a fastener of the invention;

FIG. 44 is a perspective view of an embodiment of an anchor of a fastener of the invention;

FIG. 45 is an elevation of an embodiment of a fastener of the invention;

FIG. 46 is an elevation of an embodiment of an anchor of a fastener of the invention;

FIG. 47 is a perspective view of an embodiment of an anchor of a fastener of the invention;

FIG. 48 is a depiction of an embodiment of a fastener of the invention and its position in relation to skin layers when implanted;

FIG. 49 is a perspective view of an embodiment of a fastener of the invention;

FIG. 50 is an elevation of the embodiment of FIG. 27 deployed in tissue;

FIG. 51 is a perspective view of an embodiment of a faster of the invention;

FIG. 52 is an elevation of the embodiment of FIG. 29 deployed in tissue; and,

FIG. 53 is a perspective view of an embodiment of a fastener of the invention.

[symbol]

FIG. 54 is a perspective view of an embodiment of a delivery device of the invention;

FIG. 55 is an exploded view of an embodiment of a device of the invention;

FIG. 56 is a cutaway view of a portion of an embodiment of a firing gun of the invention;

FIG. 57A is a closeup of an embodiment of a firing mechanism acting on an embodiment of a fastener of the invention;

FIG. 57B is a closeup of an embodiment of a firing pin of the invention acting on an embodiment of a fastener of the invention;

FIG. 58A is a step in a sequence of steps taken by a firing mechanism of the invention;

FIG. 58B is a step in a sequence of steps taken by a firing mechanism of the invention;

FIG. 58C is a step in a sequence of steps taken by a firing mechanism of the invention;

FIG. 59 is a perspective view of a disposable tip of the invention.

FIG. 60 is a perspective view of an embodiment of a delivery device of the invention;

FIG. 61 is a step in the use of an embodiment of a delivery device of the invention;

FIG. 62 is a step in the use of an embodiment of a delivery device of the invention;

FIG. 63 is a step in the use of an embodiment of a delivery device of the invention;

FIG. 64 is a step in the use of an embodiment of a delivery device of the invention;

FIG. 65 is a step in the use of an embodiment of a delivery device of the invention;

FIG. 66 is a step in the use of an embodiment of a delivery device of the invention; and,

FIG. 67 is a step in the use of an embodiment of a delivery device of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

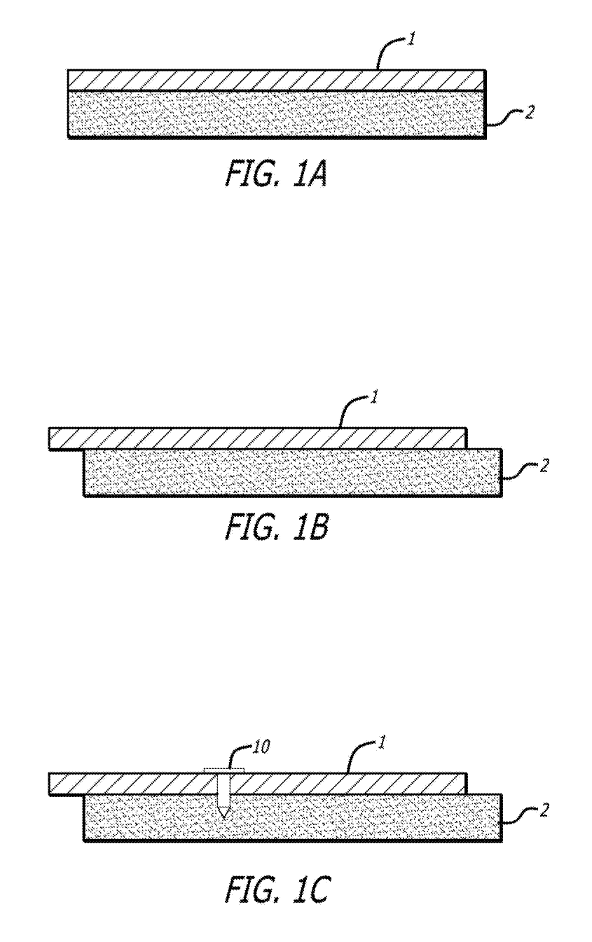

Referring now to the Figs., FIG. 1 illustrates the general method of the present invention. As shown in FIG. 1A, the method begins with a selection of a skin layer 1 to be relocated relative to a bone or cartilage layer 2. In FIG. 1B, the skin layer 1 has been shifted relative to the bone layer 2. No connective tissue between the skin layer 1 and the bone layer 2 has been severed or damaged. The tissue 1 has simply been relocated using gentle pressure, such as by a finger. Most procedures will attain desirable results with only a small shifting, akin to taking one's finger and moving one's forehead skin around gently. It is noted that the discontinuity in the edges of the tissue sample shown in FIG. 1B are simply provided for illustrative purposes to show that the skin layer 1 has been shifted relative to the bone layer 2 and is not to be interpreted as a severing or slicing of the selected layer 1. In actuality, the continuous skin layer 1 stretches, and thus releasing the skin layer 1 would result in its return to the original position shown in FIG. 1A.

In order to prevent the return of the skin layer 1 to its original position relative to the bone or cartilage layer 2 of FIG. 1A, a fastener 10 of the present invention is used to affix the skin layer 1 to the bone or cartilage layer 2 at its new position. This step is shown in FIG. 1C. The fastener 10 is shown as a simply tack-like device in FIG. 1C. Various embodiments of fastener 10 will be shown and described below.

The method described above and shown in FIG. 1 is a simple illustration of the basic concepts of the present invention. It is envisioned that the steps of shifting the skin and anchoring it a new position will be repeated many times until a desired result is achieved. It is also likely that a single shift will be followed by multiple fasteners 10 be applied to secure a given shift of the skin layer 1. Subsequent shifts will then likely be required in order to achieve a desired, natural-looking result.

In one embodiment of the method of the present invention, extremely small, short and shallow incisions may be made at the site where the fastener 10 is being driven such that the head or top of the fastener rests just below the surface of the skin such that the fastener remains hidden. These incisions are so small that often no bleeding occurs. Positive results have been attained with incisions that are no longer than 0.05 inches.

In order to accommodate the insertion of several fasteners 10, various delivery mechanisms are described below, some of which allow the delivery of rapidly successive fasteners, in the spirit of tools found in carpentry, such as nail guns, staple guns, and the like. Other delivery mechanism described below allow for the delivery of several fasteners simultaneously.

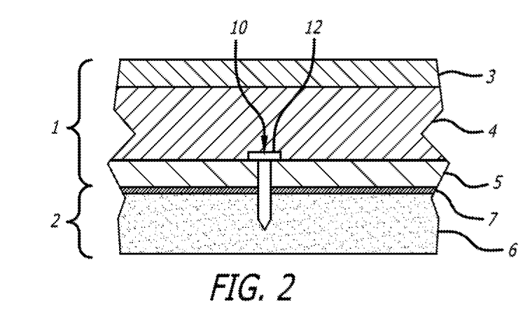

FIG. 2 provides a more detailed view of the skin layer 1, the bone or cartilage layer 2 and the device 10. The skin layer 1 comprises an epidermis 3, a dermis 4 and a subcutaneous layer 5. Superficial musculoaponeurotic system (SMAS) fascia is a fanlike fascia that envelops the face and provides a suspensory sheet which distributes forces of facial expression. On a cellular level it comprised of collagen fibers, elastic fibers, fat cells and muscle fibers.

The bone layer 2 includes the bone 6 and a periosteum 7. It can be seen in FIG. 2 that the fastener 10 is implanted such that it penetrates the bone 6 and the periosteum 7 and the proximal end 12 of the fastener 10 terminates within the subcutaneous layer 5, or the dermis 4. In this way, the fastener 10 remains invisible once implanted. The SMAS layer is the structure upon all modern, traditional facelifts are based. Traditional facelift addresses the lower face and neck. It involves an incision made in the hairline, starting above the ear, continuing behind the ear, curving around the ear and ending in the hairline behind the ear. The surgeon dissects the skin from the underlying fat and muscle. He will then use sutures to lift and reposition the muscle layer (“SMAS”—superficial musculo-aponeurotic system) toward the ears. This muscle tightening is thought to provide longevity to the surgical result. Next, the excess skin is removed and the incisions are closed.

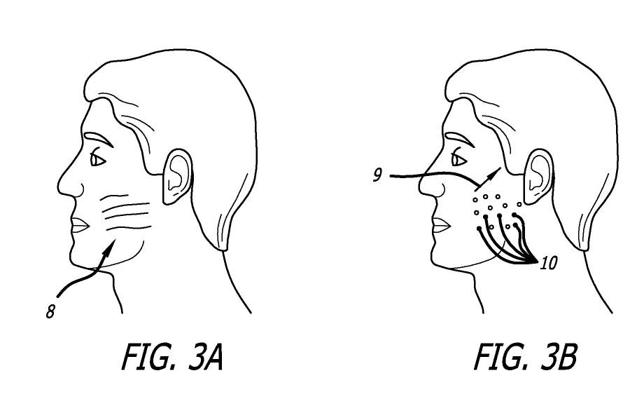

FIG. 3 shows the general effect of correcting the appearance of wrinkles 8 on the face (FIG. 3A) through the use of multiple anchors 10 to hold the soft tissue in place in a new position after the face has been shifted in the direction of arrow 9 (FIG. 3B).

### Fasteners

Turning now to the fasteners 10 of the invention, the fasteners 10 may take one of many possible forms. Generally, they may be circular, flat, or any other configuration geometrically that allows them to penetrate the bone or cartilage with a sharpened distal end. The device may be textured on the surface, for example with a micro-texturing that allows cells to more easily attach and anchors the device permanently in the bone or cartilage. The anchors may be metallic or they may be polymeric. They may be a combination of metal and polymer. The polymer may be biostable or bioabsorbable. It may contain drugs for elution. The anchors may be electrically conductive and may permit electrical energy for either energy delivery or energy recording of biologic signals. Examples of embodiments having various characteristics are shown in the Figs. and are not meant to be limiting. It is to be understood that any of the characteristics may be incorporated into any of the embodiments of the invention.

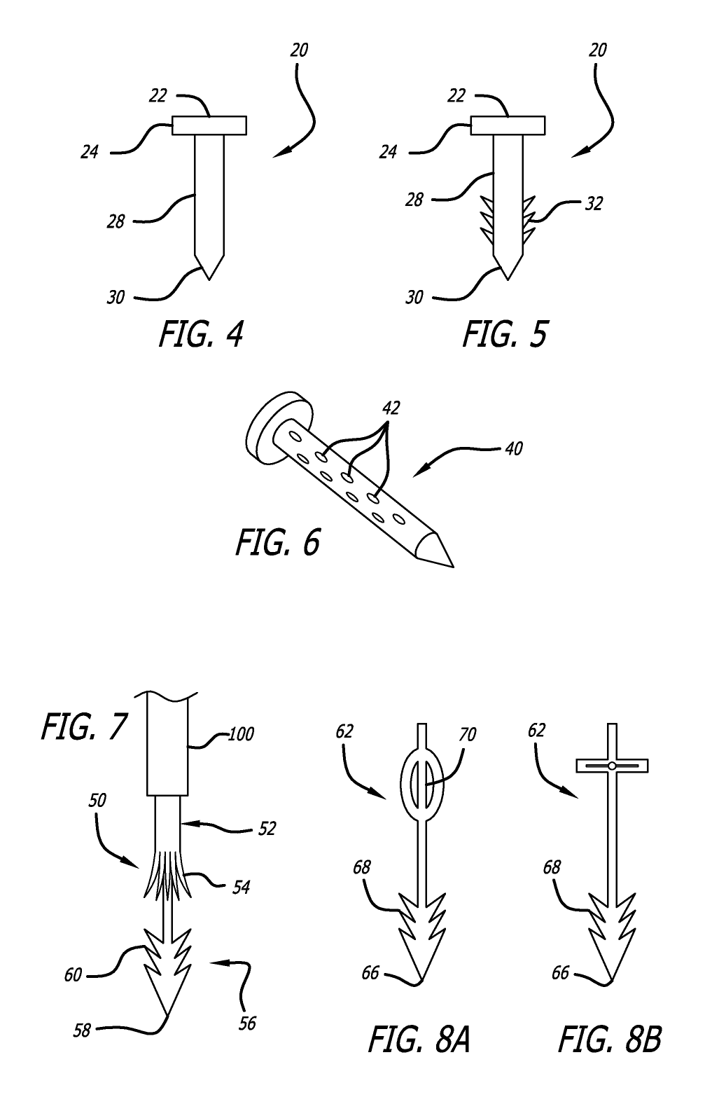

Generally, the fasteners include at least one shaft that includes an anchoring feature proximate the distal end of the shaft and a tissue-holding feature proximate the proximal end of the shaft. A first embodiment 20 of a fastener 10 is shown in FIG. 4. This embodiment 20 includes a head 22 as a tissue-holding feature at a proximal end 24 of the anchor 20. The head 22 may be circular and generally flat for pushing or hammering. A shaft 28 is provided with sufficient length to anchor the fastener 20 deep enough into the bone to resist becoming dislodged, while still terminating such that the head results at a desired depth in the skin layer 1. The distal end of the shaft presents an anchoring feature that includes a sharpened distal tip 30 that allows the device 20 to be driven through the skin layer 1 into the bone layer 2. As shown in FIG. 5, the anchoring feature may also include features 32 that prevent the device 20 from migrating proximally after having been implanted. These features 32 may comprise hooks, barbs, ridges, high-friction surfaces, in-growth inducing coatings, adhesives, and the like.

FIG. 6 shows an embodiment 40 in which the fastener 10 has a hollow shaft that includes apertures 42 that lead to an interior lumen that may contain a drug or agent. The drugs may elute out slowly or rapidly and may be initially contained within the lumen, or may be injected through the lumen after the device is implanted. The drug may have a polymer component to it to program the rate of delivery. The drug may be in the form of nanoparticles that are driven into the bone or leach out into the bone or cartilage at a programmable rate.

FIG. 7 shows an embodiment of a fastener 50 that includes plungers 52 and 54 that engage the tissue and anchor the tissue to the bone or cartilage. In FIG. 7, the device 50 is being deployed from a delivery device 100. The device 50 includes a tissue-holding feature shown as a first plunger 52 having a plurality of fingers 54 that splay outwardly as they exit the delivery device 100 and engage tissue of the skin layer 1. A second plunger 56 having a sharp distal tip 58 and anchoring features 60 extends partly through the first plunger and is designed to penetrate the bone layer 2 and remain anchored therein.

FIGS. 8A and 8B show a fastener embodiment 62 in which the two plungers are connected as one device. In embodiment 62, the device includes a distal end 64 with a sharpened tip 66 and anchoring features 68, similar to those of the embodiment 50 of FIG. 7. The tissue-holding feature at the proximal end of the device 62, however, includes a plurality of slits 70 that, when compressed or foreshortened, flare outwardly to engage tissue, as seen in FIG. 8B.

The slots 70 may be actively flared in a subsequent step, however, it is envisioned that such slots 70 may be advantageously paired with one or more of the ballisitic delivery device embodiments, described below. When the device 10 is driven into the bone layer, there is substantial compression on the device that causes the precut slots 70 to expand radially, markedly increasing the cross-sectional area of a small section of the anchor. When placed appropriately, this expanding tissue-holding concept will serve to grasp the soft tissue ends spreading the stress and strain on the tissue out over a larger area to prevent laceration. It will serve as a larger tissue-holding feature for the soft tissue because its surface area in contact with the soft tissue is substantially increased.

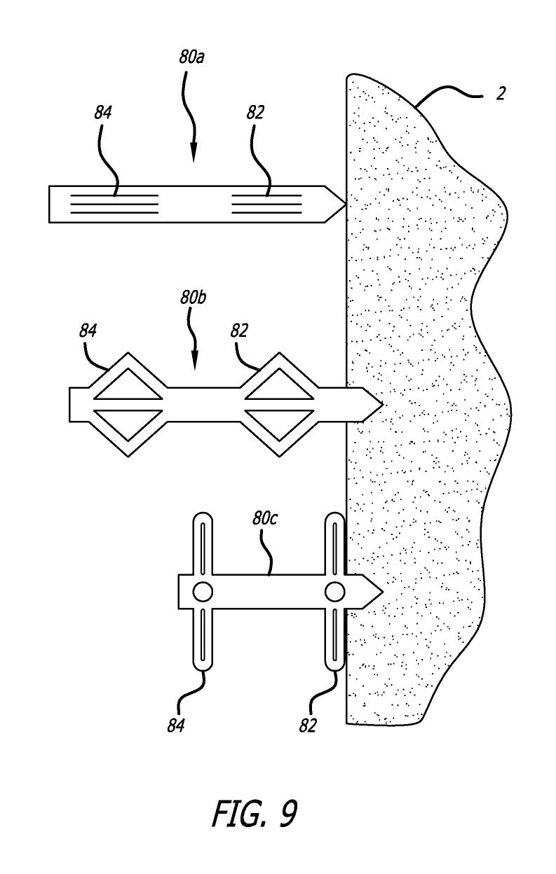

FIG. 9 shows a fastener embodiment 80 incorporating a distal set of slots 82 and a proximal set of slots 84. Three fasteners 80 are shown in various stages of implantation. Device 80a has not yet encountered the bone layer 2 and is therefore not yet subject to compressive force. Device 80b has contacted the bone layer 2 and, due to the compressive force provided by the resistance of the bone layer 2, the slots 82 and 84 begin to bend outwardly. Device 80c is completely driven into the bone layer 2 and both sets of slots 82 and 84 are completely flared. The distal set 82 forms a stop that prevents the device 80 from travelling further into the bone layer 2. The proximal set 84 forms a tissue-holding feature that engages tissue from the tissue layer 1, thereby preventing the tissue layer 1 from sliding over the device 80 and adjusting itself to a previous position. If it is desired to further increase the tissue holding power of the proximal slots 84, additional features such as barbs or hooks could be incorporated into the device 80. Advantageously, this configuration allows a small diameter device 80 to be inserted through the skin, while expansion does not occur until the device reaches the dermal or subcutaneous layer, thereby maximizing holding power while minimizing trauma to the visible entry point, thus promoting quick healing.

The resulting arms formed by the slots may be retractable in the event that the operator is unhappy or unsatisfied with placement, and re-deployed for better configurations and results. Additionally, the above describes only two locations on the anchor where the “stop” components are placed, but multiple other locations may be made as well to further anchor in the soft tissue.

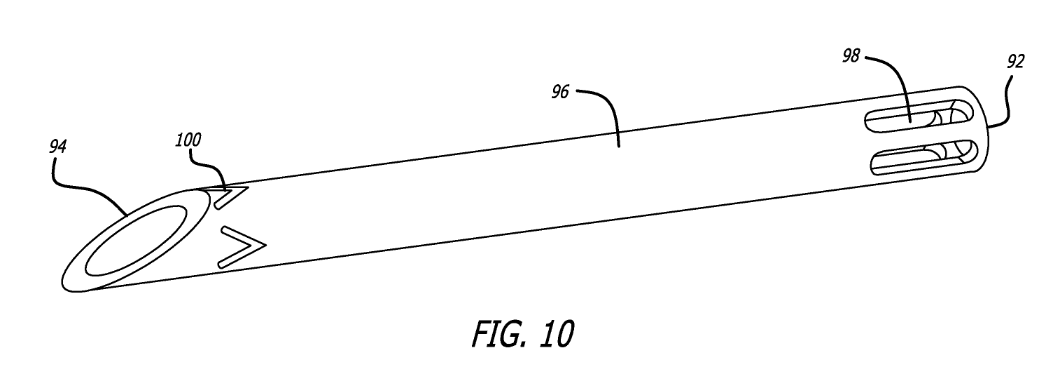

FIG. 10 shows another fastener embodiment 90. Fastener 90 includes a proximal end 92, a distal end 94 and a shaft 96. The proximal end 92 includes a plurality of slots 98, similar to the slots 84 of embodiment 80, described above, in both form and function. Slots 98 allow the material between the slots to flare outwardly to form a tissue-holding feature. The distal end 94 is sharpened at an angle to allow the fastener to pierce bone tissue. An anchoring feature 100 includes a plurality of angled cut-outs that flare outwardly when driven into the bone for grabbing the bone layer. The cut-outs may flare outwardly due to the force of being driven into the bone or they may be formed of a memory metal or they may be manufactured with outward flares. The embodiment 96 is also shown has having a hollow shaft 96 with an open distal end. It has been found that a hollow shaft with an open distal end results in a fastener that is easier to drive into the bone, as less bone material needs to be displaced.

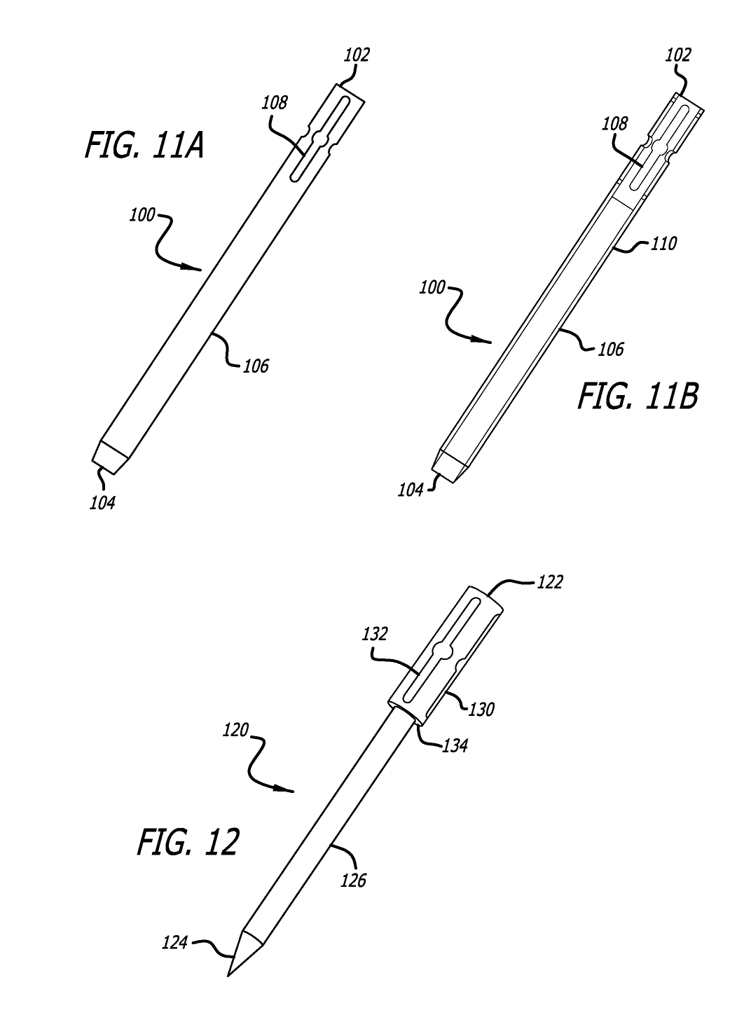

FIGS. 11A and 11B show a fastener embodiment 100 that includes a proximal end 102, a distal end 104 and a shaft 106. The proximal end 102 includes a plurality of slots 108, similar to the slots 84 of embodiment 80, described above, in both form and function. Slots 108 allow the material between the slots to flare outwardly to form a tissue-holding feature. Like fastener 90, the shaft 106 is also hollow. As best seen in FIG. 11B, which is depicted as translucent to show the interior features of the device 100, the interior lumen of the shaft 106 terminates proximally at a stop 110. Stop 110 limits the depth that the fastener 100 can be driven into the bone layer. The stop 110 also prevents inward deflection of the tissue-holding feature formed by the slots 108.

The distal tip 104 of fastener 100 is also sharpened. However, rather than being sharpened at an angle like fastener 90, the distal tip 104 of fastener 100 is sharpened circumferentially to form a beveled edge. It has been found that a tip sharpened this way penetrates bone more easily, possibly due to the cancelation of all side forces resulting from the angled surfaces. Additionally, the angled tip of fastener 90 has a rapidly-increasing surface area as it is being driven into bone. The beveled tip, on the other hand, as a relatively constant surface area contacting the bone as it is being driven. Experimentation has shown that holding all other variables constant, driving 5 mm long fasteners with angled tips into a bone layer with a constant driving force resulted in a driving depth of 2-3 mm. When driving 5 mm fasteners with beveled tips using the same driving force, the driving depth was approximately 4 mm.

It must be emphasized that any of the features described herein with regard to one embodiment may be combined with any of the features of the other embodiments.

FIG. 12 shows yet another fastener embodiment 120. Fastener 120 has a shaft 126 with a proximal end 122, and a distal end 124. The shaft 124 of fastener 120 is shown, by way of example, as being solid and sharpened to a point. The proximal end 122 of the fastener 120 has a tissue-holding feature 130 with a plurality of slots 132 that function in a similar fashion to the slots described above in association with the various other embodiments. The tissue-holding feature 130, however, is shown as having an increased radius in relation to the shaft 126. This is because the shaft 126 is solid, so forming slots in a solid shaft would not result in flaring when the shaft is impacted. Additionally, a distal edge 134 of the tissue-holding feature 130 forms a stop that limits the depth the fastener 120 can be driven into a bone layer. The distal edge 132 also causes outward deflection of the tissue-holding feature formed by the slots 134.

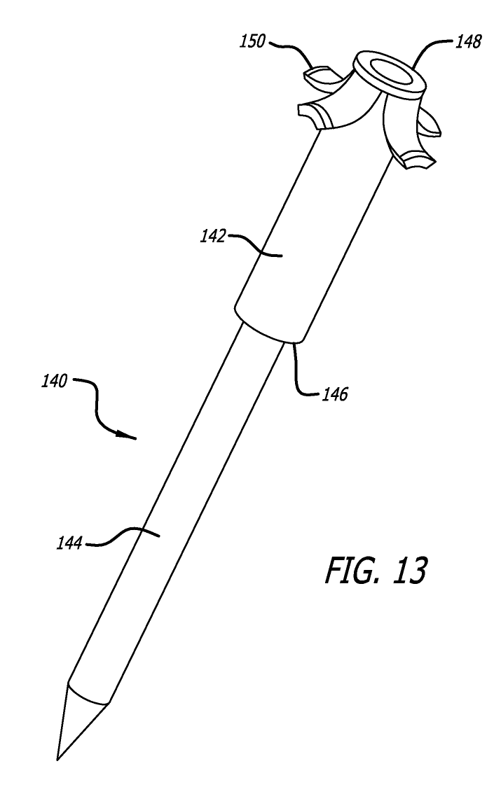

FIGS. 13-17 show various fastener embodiments utilizing different tissue-holding features. For example, FIG. 13 shows a fastener embodiment 140 with a sliding collar 142 that slides over the shaft 144 of the fastener 140. The collar 142 has a distal edge 146 that acts as a stop to limit the depth that the fastener 140 can be driven into a bone layer. The edge 146 also causes the collar 142 to slide proximally over the shaft 144 when the edge 146 contacts the bone layer. Sliding proximally over the shaft 144 causes a proximal edge 148 of the collar to impact and deform a plurality of wings 150. The wings 150 are deformed to splay outwardly, thereby creating a tissue-holding feature.

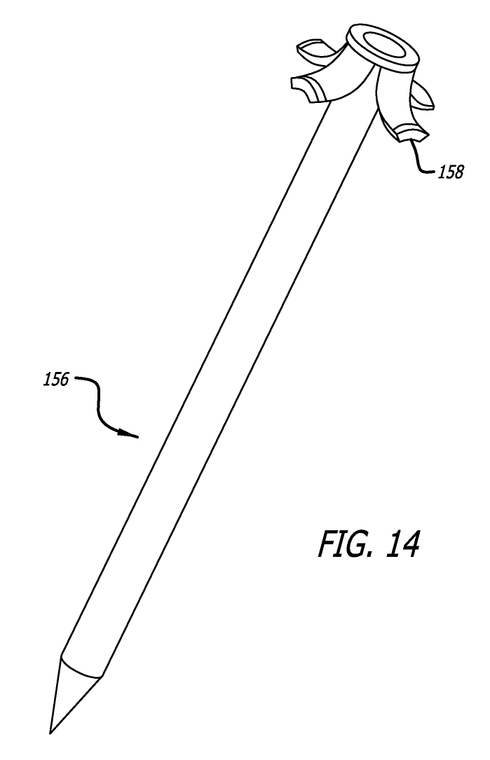

FIG. 14 shows a fastener embodiment 156 having a similar tissue-holding feature including a plurality of outwardly-splayed wings 158. These wings 158, however, are formed of a memory metal such that they splay without requiring impact forces.

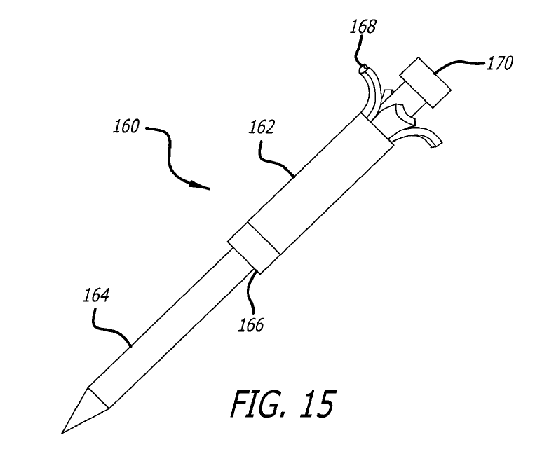

FIG. 15 shows a fastener embodiment 160 with a sliding collar 162 that slides over a shaft 164 of the fastener 160. The embodiment 160 also includes a fixed collar 166 that has a distal edge that acts as a stop to limit the depth that the fastener 160 can be driven into a bone layer. The sliding collar 162 has a plurality of wings 168 at its proximal end. The shaft 164 has a proximal fixed collar 170 at its proximal end, which keeps the sliding collar 162 from sliding too far proximally. The wings 168 splay are made from nitinol or a similar memory metal and splay outwardly when delivered to form a tissue-holding feature.

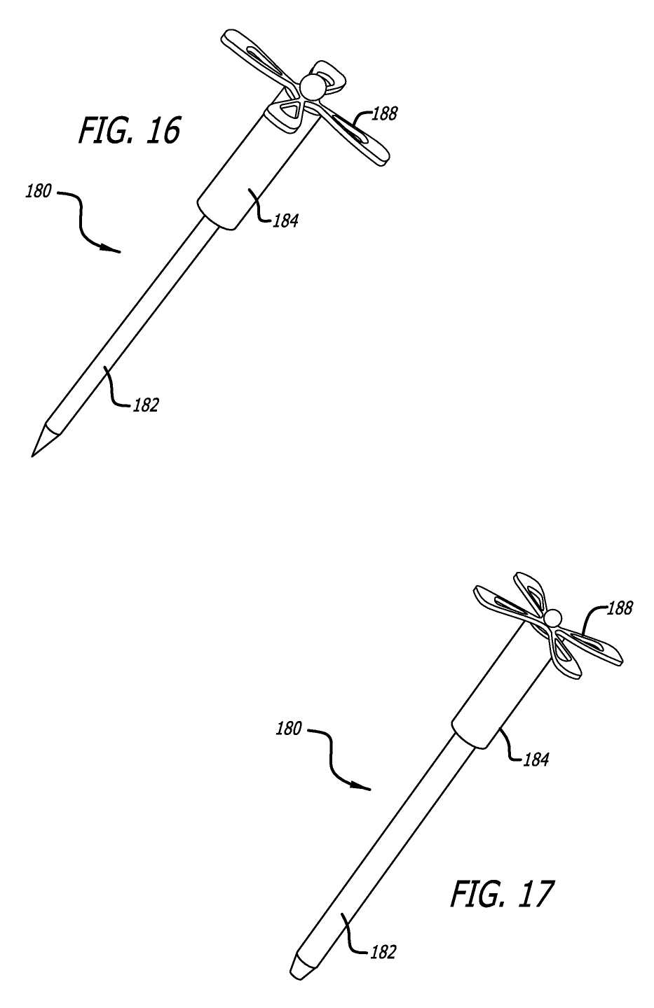

FIG. 16 shows a fastener embodiment 180 with a shaft 182 and a collar 184 having a distal edge 186 that acts as a stop. The fastener 180 includes as a tissue-holding feature a plurality of petals 188 at its proximal end. The petals 188 are flat and radiate outwardly, perpendicular to a longitudinal axis of the shaft 182. The petals 188 may be formed to splay outwardly by way of the collar 184, as described above, or the petals 188 may be formed of memory material. Additionally, the petals 188 may be stored in a delivery device in a proximally or distally directed fashion.

FIG. 17 demonstrates that the shaft 182 of the fastener 180 may be hollow and that each of the embodiments shown has features that may be combined with features shown in other embodiments.

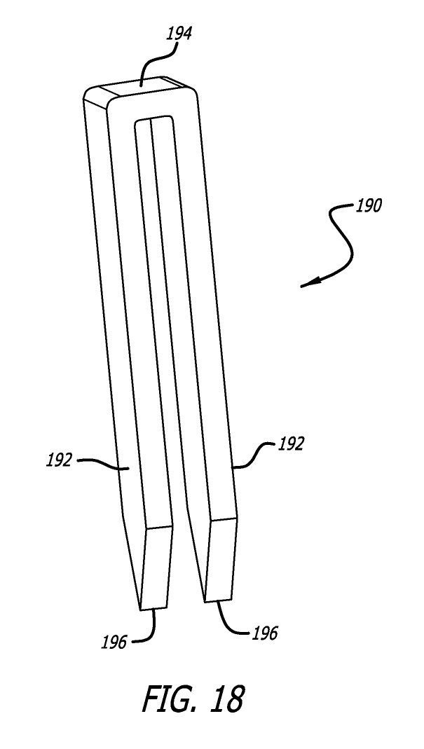

FIG. 18 shows a fastener embodiment 190 that includes two shafts 192 connected at their proximal ends with a bridge 194, thus forming a staple. The bridge 194 serves as a tissue-holding feature. Each of the shafts 192 includes a pointed distal tip 196 that allows the fastener 190 to be driven into bone. The shafts 192 serve as anchoring features.

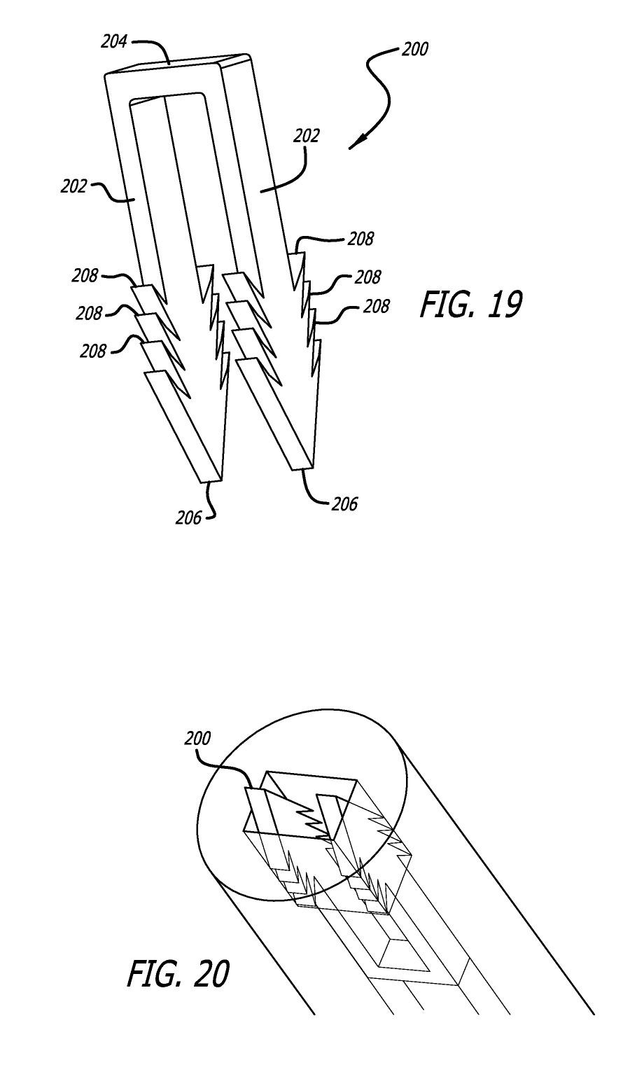

FIG. 19 shows a fastener embodiment 200 that includes two shafts 202 connected at their proximal ends with a bridge 204, thus forming a staple. Like that of FIG. 18, the bridge 204 serves as a tissue-holding feature. Each of the shafts 202 includes a pointed distal tip 206 that allows the fastener 200 to be driven into bone. The shafts 202 also include anchoring features in the form of barbs 208. FIG. 20 shows the fastener 200 loaded into the distal end of a delivery device. The various embodiments of delivery devices are discussed in more detail below.

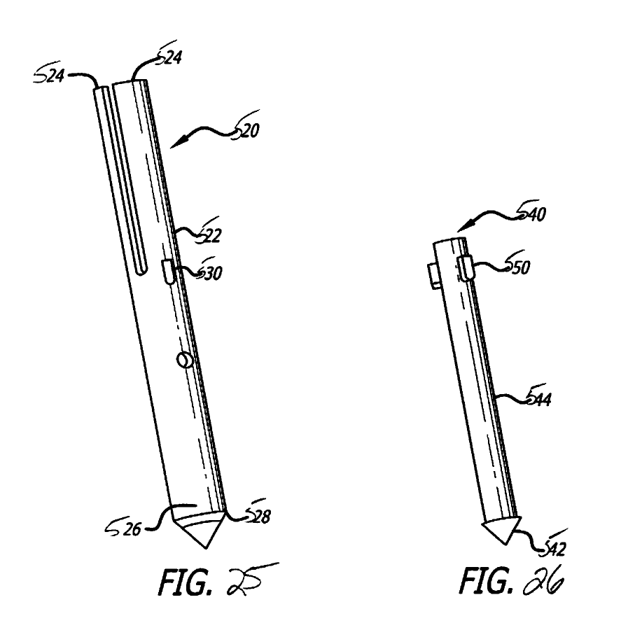

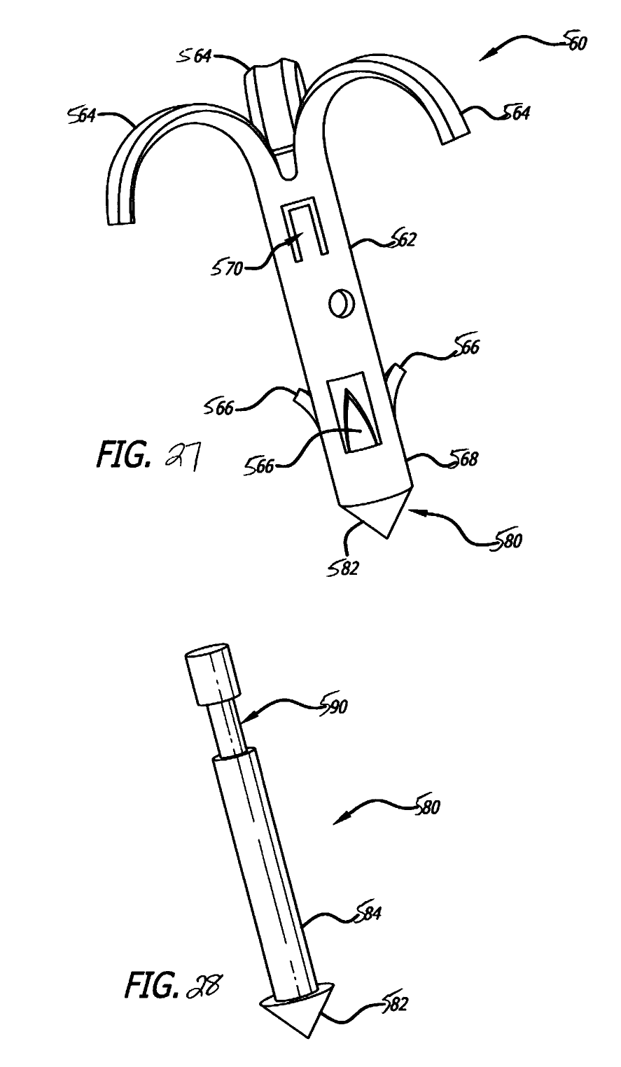

Generally, the fasteners may include one or more shafts and also include an anchor proximate one end of the shaft and a tissue-holding feature, hereinafter “tissue holder,” proximate the other end of the shaft. Another embodiment 520 of an fastener 10 is shown in FIG. 25. This fastener 520 includes a fastener 522 and a spike 540 (FIG. 26) that may be contained within the fastener 522.

The fastener 522 may be a memory metal, such as Nitinol, that includes soft tissue anchoring features that expand or flare outwardly. Alternatively, these features may be made of other materials and flare upon impact with the bone layer (in-situ formation of the feature). The fastener shown in FIG. 25 includes tissue holders 524 and distal anchors 526. The tissue holders 524 are in the form of elongate petals that are formed by cutting slits in the tubular fastener 522. The elongate petals 524 curl outwardly upon deployment, catching the fibrous tissue layer.

The distal anchoring features 526 comprise barbs that flare radially upon implantation. These barbs 526 are generally smaller than the petals 524 as they are designed to imbed themselves in bone rather than soft tissue. The distal anchoring features 526 of FIG. 25 are shown as circumferentially-formed features. It is anticipated that the device may work sufficiently without the distal features (barbs), but that they may aid in long term securement.

The distal end 528 of the fastener 522 may be beveled to match a slope of the distal end of the spike 40, discussed below. Additionally, the fastener 522 may include an aperture 530 for receiving a locking feature, such as a protuberance 550, on the spike 540.

The spike 540 assists in driving the fastener 522 into the bone layer. To accomplish this assistance, the spike 540 includes a sharpened distal end 542 and a solid shaft 544. The spike 540 may be formed of a hardened material such as stainless steel or Titanium. The solid shaft 544 provides column strength, and prevents the hollow shaft of the fastener 520 from bending or otherwise collapsing upon impact with either the driving mechanism or the bone. The spike 540 shown in FIG. 26 includes a locking feature 550 in the form of a groove, notch or protuberance. The locking feature 550 ensures that the spike 540 and the fastener 520 act in unison when being driven into the bone. Alternatively, the spike 540 and fastener 520 could be welded, glued or otherwise attached to each other. Alternatively the spike 540 and fastener 520 could be fashioned from a single piece of material. Alternatively the spike 540 could be temporary and removed after the fastener 520 is in place.

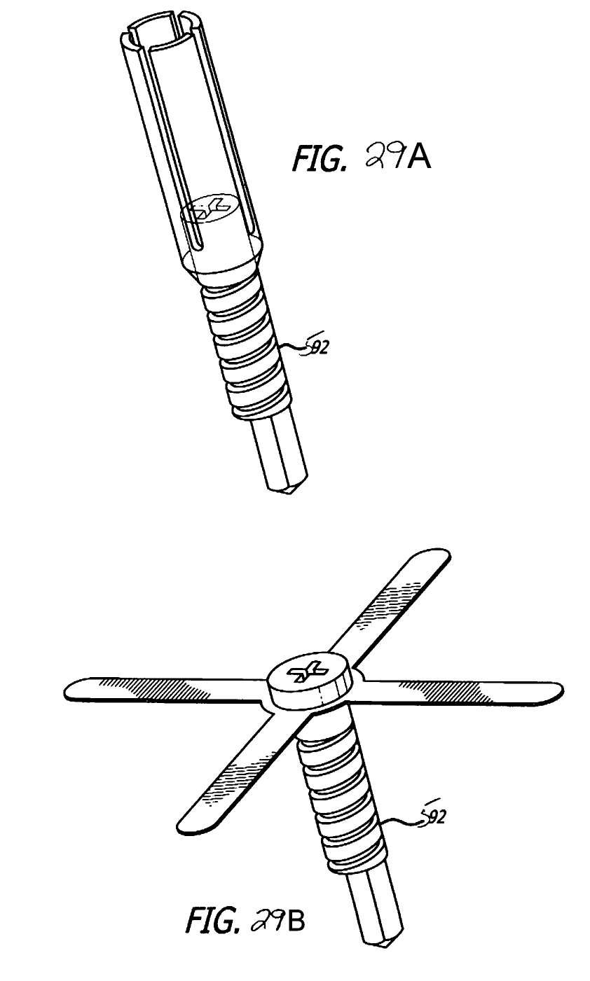

FIGS. 27 and 28 shows another embodiment 560 of a fastener 562 and a spike 580. The fastener 562 includes tissue holders 564 and distal anchors 566. The tissue holders 564 are in the same form of elongate petals as the features 524 shown in FIG. 25, except that the holders 564 are shown in the expanded state. The elongate petals are loaded in a straight configuration but curl outwardly upon deployment, securing the soft tissue of the dermis and/or fibrous tissue layers.

The distal anchors 566 comprise barbs that flare radially upon implantation. These barbs 566 are smaller than the petals 564 as they are design to imbed themselves in bone rather than soft tissue. The distal anchors 566 of FIG. 27 are shown as longitudinally-formed features.

The distal end 568 of the fastener 562 is flat to act against a corresponding feature of the distal end of the spike 580, discussed below. Additionally, the fastener 562 may include a tab locking feature 570 that is inwardly biased to engage a corresponding recess 590 in the spike 580.

The spike 580 assists in driving the fastener 562 into the bone layer. To accomplish this assistance, the spike 580 includes a sharpened distal end 582 and a solid shaft 584. The sharpened distal end 582 is slightly larger than the diameter of the fastener 562. The enlarged end 582 creates a larger hole in the bone, which facilitates the driving of the fastener 562 into the bone material. Because the enlarged end 582 is slightly larger than the diameter of the fastener 562, the end 582 acts as another anchor for the fastener 560.

The spike 580 may be formed of a hardened material such as stainless steel or Titanium. The solid shaft 584 prevents the hollow shaft of the fastener 562 from bending or otherwise collapsing upon impact with either the driving mechanism or the bone. The spike 580 shown in FIG. 28 includes a recess 590 that is engaged by the tab locking feature 570 of the fastener 562. The locking feature 570 ensures that the spike 580 and the fastener 562 act in unison when being driven into the bone.

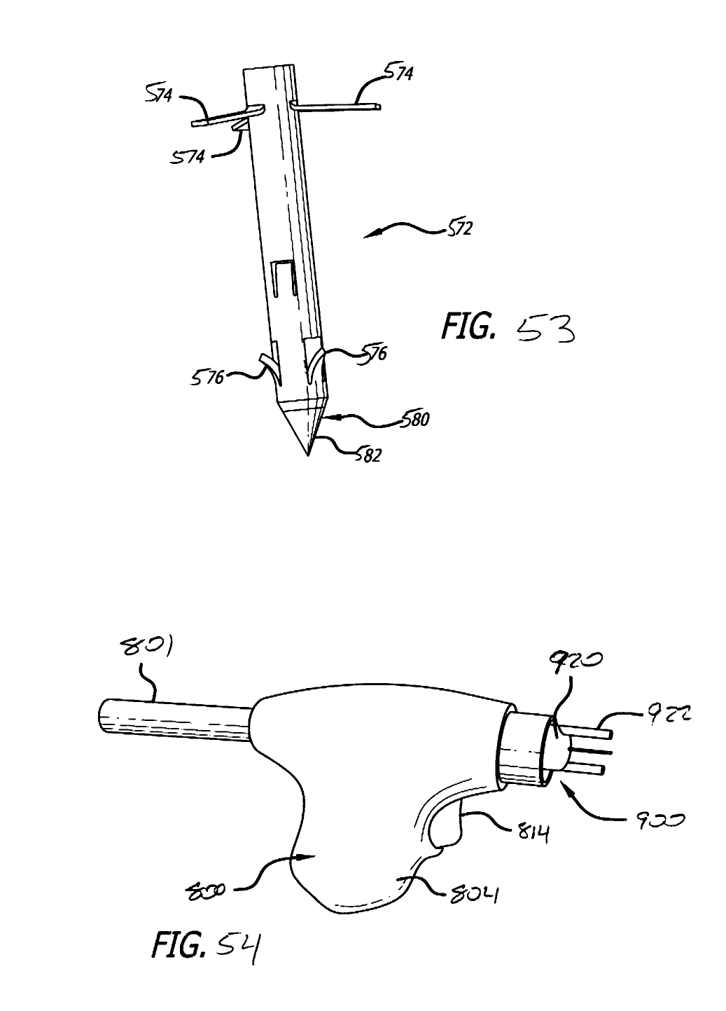

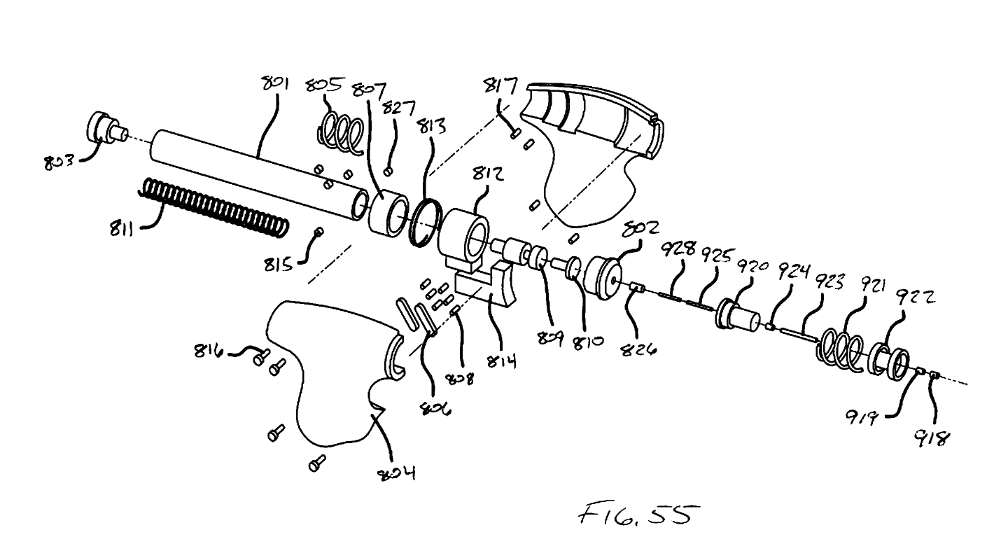

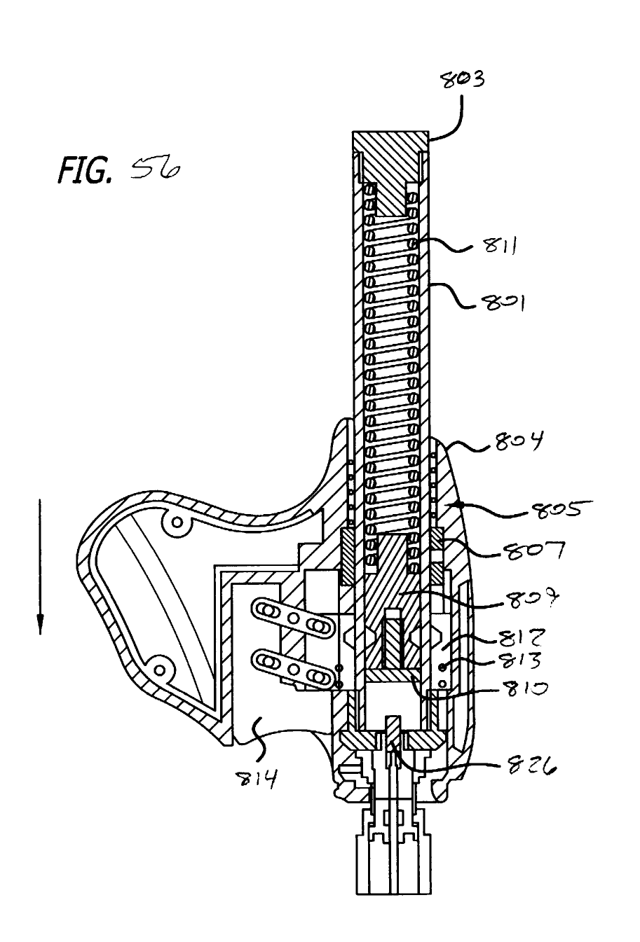

Fastener 562 is shown as having tissue holders 564 that curl distally, toward the anchors 566. However, an embodiment of a fastener 572, shown in FIG. 53, includes tissue holders 574 that flare proximally, away from anchors 576. The fastener 572 may be used with spike 580.

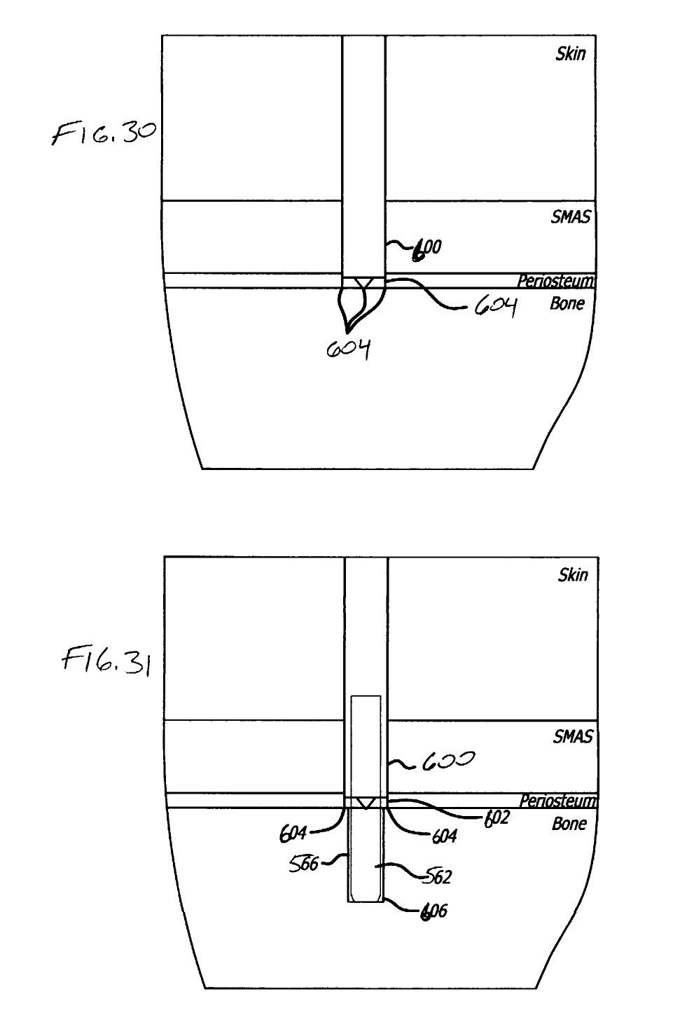

FIGS. 29A and 29B show an alternative spike design in the form of a screw 592. The screw 592 may be used in conjunction with a predrilled hole in the bone or may be self-tapping. The screw 592 is attached to a fastener such that the fastener is carried with the screw 592. FIGS. 29A and 29B show the screw 592 being used in conjunction with a fastener 10.

FIGS. 30-32 show a delivery sequence for a fastener 10. Beginning with FIG. 30, a small incision is made and then a blunt dissection is performed with a cannula 600 down to the surface of the bone layer. The tip 602 of the cannula 600 may have protrusions 604 that slightly penetrate into the bone to aid in fixating the cannula 600 in a desired position relative to the bone.

In FIG. 31, a hole 106 is pre-drilled into the bone. The hole is slightly oversized such that the fastener 562 is easily placed into the hole. It can be seen that the fastener 562 has anchors in the form of barbs 566 that flare outwardly to anchor the fastener 562 into the bone. Many of the fastener embodiments described herein have sharpened distal tips, such as those of FIGS. 25-27, and would not require this pre-drilling step, as they are able to be driven directly into the bone.

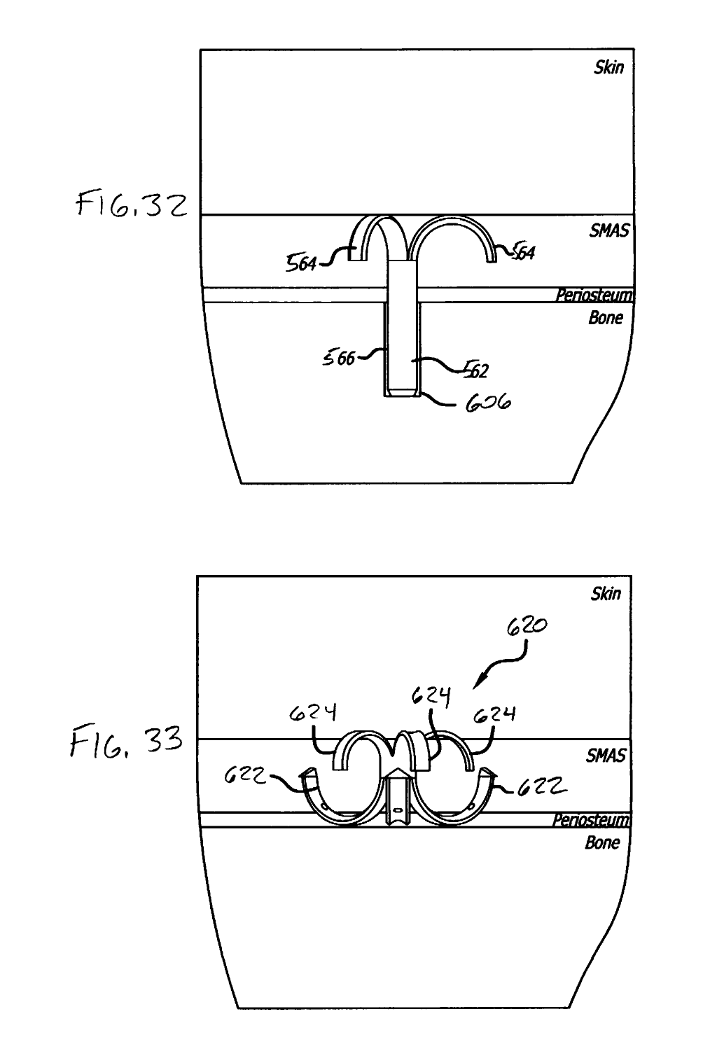

FIG. 32 shows that the cannula 600 has been removed and the fastener 562 remains. The fastener 562 has tissue holders in the form of petals 564 that have curled outwardly, through the soft tissue, thereby capturing the fibrous tissue layer.

FIG. 33 shows a double-petal design for a fastener 620. The fastener 620 has anchors in the form of distal petals 622 and tissue holders in the form of proximal petals 624. The distal petals 622 penetrate only through the periosteum (Pericranium), but do not penetrate into the bone, thus accomplishing fixation by penetrating only the relatively strong and in-elastic pereosteum.

The proximal petals 624 secure the deep dermal tissue and/or fascia layers. This embodiment exhibits greater ease in deployment and removal, if necessary. It has also been demonstrated that these distal petals can be designed to penetrate the bone itself to anchor the device.

It must be emphasized that any of the features described herein with regard to one embodiment may be combined with any of the features of the other embodiments. It is further to be understood that the terms “anchors” and “tissue holders” are being used to distinguish the deeper anchor features from the shallower anchor features. Both the anchors and the tissue holders could be described as functioning as “anchors” or as “tissue holders”. As such the names given to each are not to be interpreted as limiting the functions of the features.

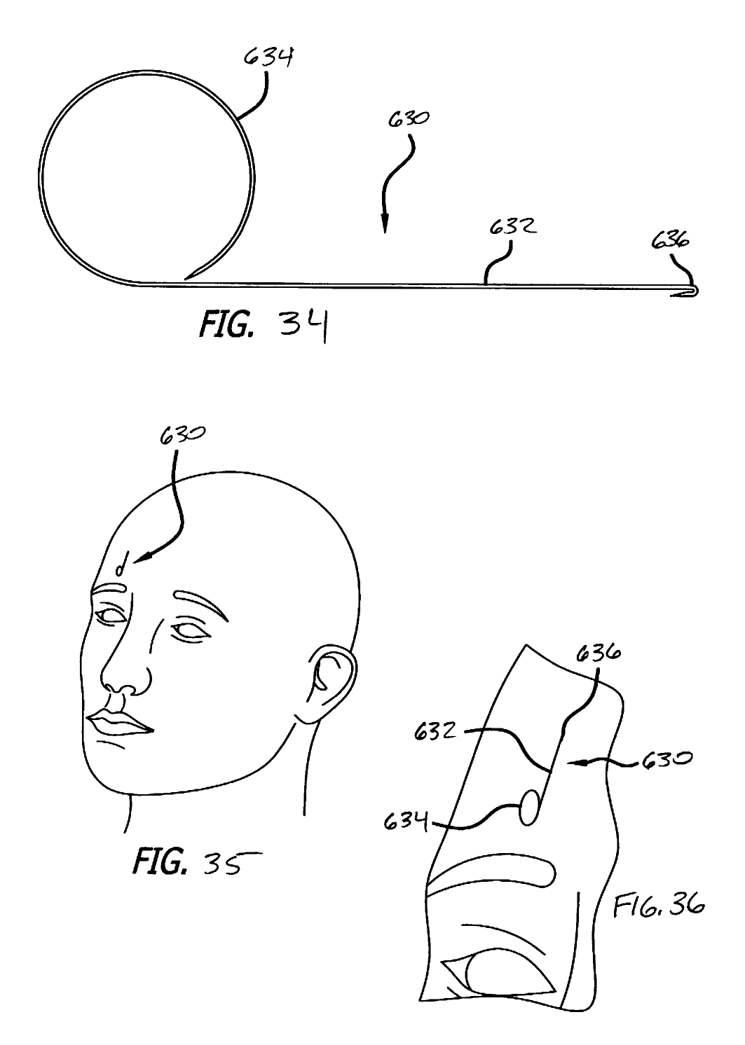

FIG. 34 shows another embodiment of a fastener 630 of the invention. Fastener 630 differs from the other fastener embodiments discussed thus far in that the fastener 630 includes a tissue holder 634 that is laterally displaced from an anchor 636 by a shaft 632 that extends substantially parallel to the bone layer, when implanted. The anchor 636 takes the form of a hook or barb that is used to fix the fastener 630 in position relative to a periosteum.

The tissue holder 634 curls up and away from the periosteum, when implanted, to engage more surface tissue layers such as the connective tissue layer. The positioning of the fastener 630 is depicted in FIGS. 35 and 36, in which the fastener 630 is shown as implanted in the forehead of a patient.

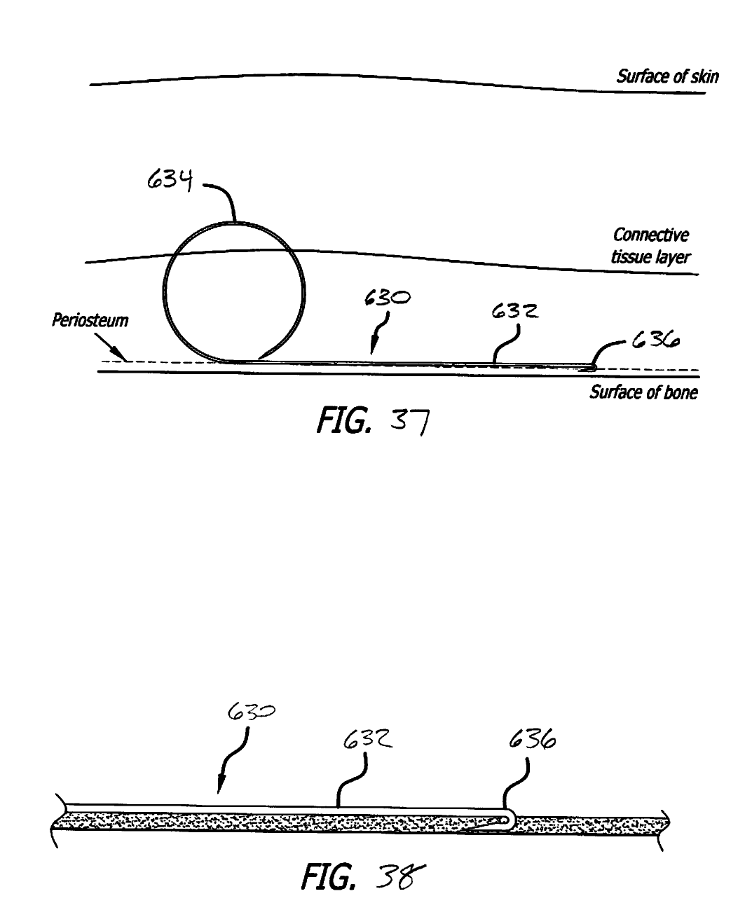

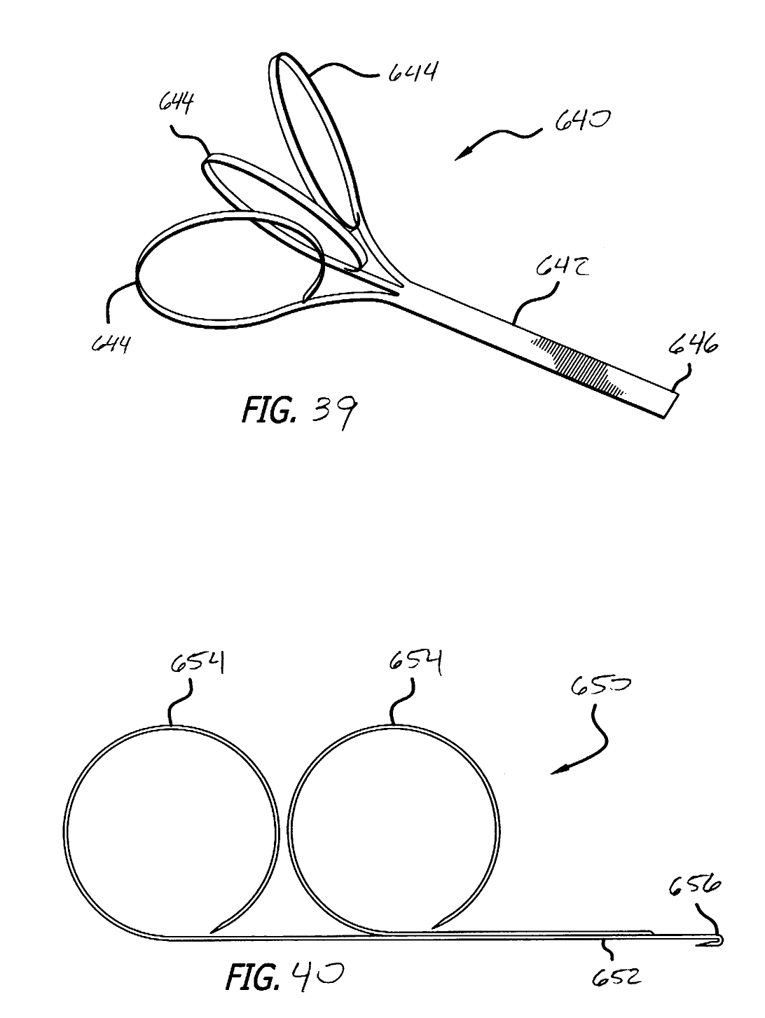

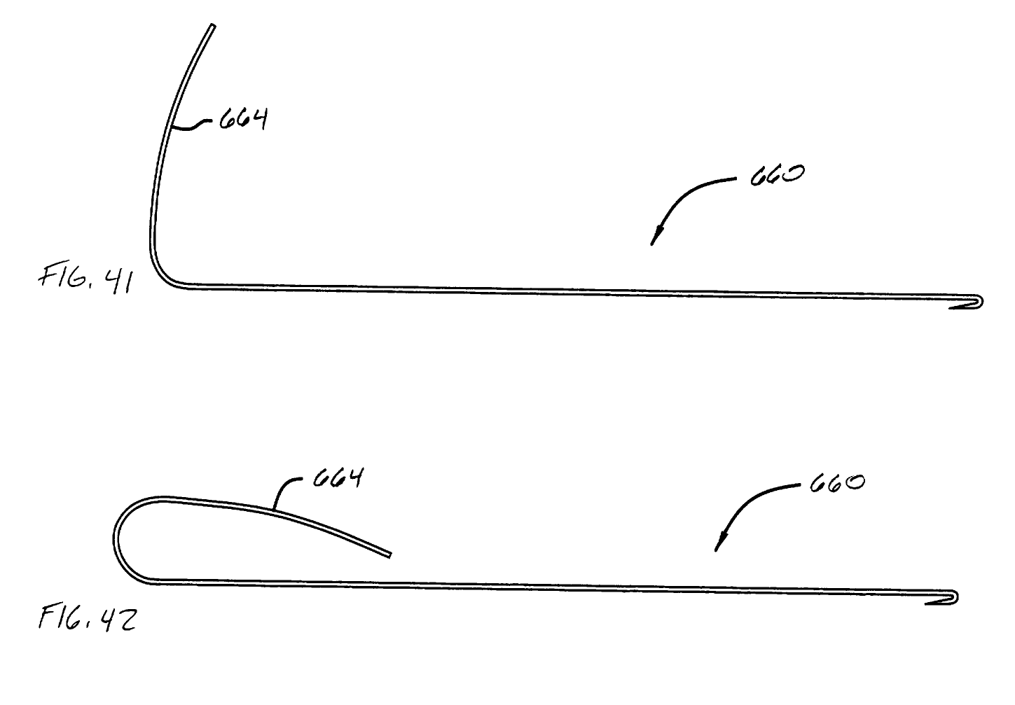

FIG. 37 shows the positioning of the fastener 630 relative to the various tissue layers. The periosteum is shown as a dotted line, just above the surface of the bone. It can be seen that the anchor 636 engages the periosteum, just above the bone layer. The shaft 632 runs substantially parallel to the bone layer. The tissue holder 634 curls away from the periosteum toward the surface of the skin, but remains below the surface. The tissue holder 634 is sized to engage the connective tissue layer without becoming exposed through the surface of the skin. FIG. 38 is a close-up depiction of the anchor 636 engaging the periosteum.

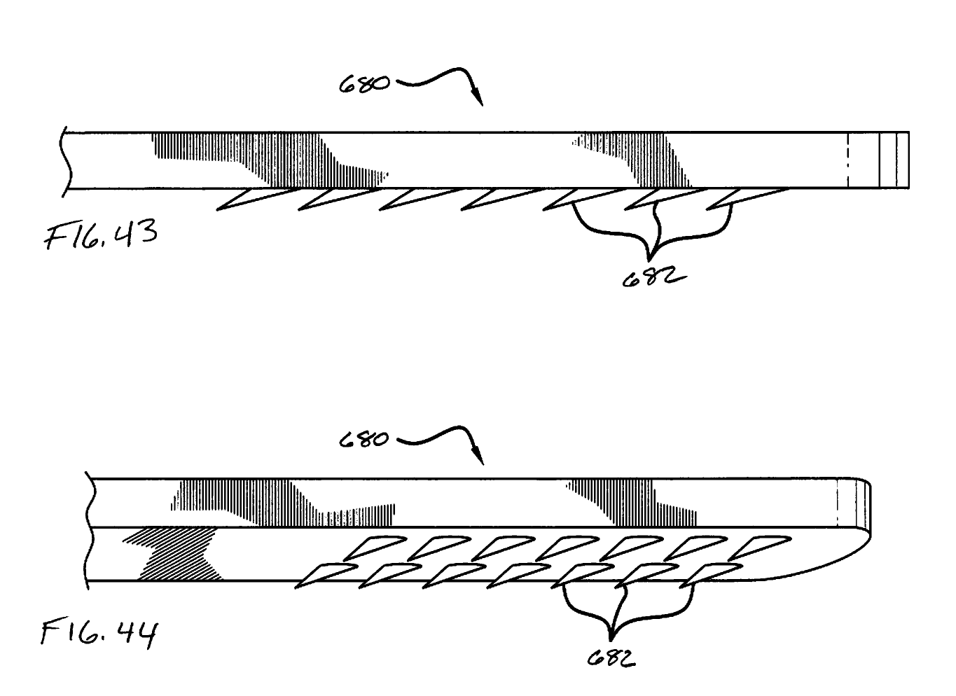

FIG. 39 shows an embodiment 640 of a fastener of the invention. Fastener 640 includes a shaft 642, a tissue holder 644 and an anchor 646. The fastener 640 is distinguished from fastener 630 by the tissue holder or holders 644. Whereas fastener 630 shows a single tissue holder 634, fastener 640 is shown as including multiple tissue holders 644. The fastener 640 is shown in FIG. 39 as including three tissue holders 644 but one skilled in the art will understand that fewer or more may be used. The tissue holders 644 are adjacent to each other and displaced by angling them away from each other.

Another embodiment of a fastener 650 is shown in FIG. 40, which also has multiple tissue holders 655. The tissue holders 655, rather than being adjacent to each other, are spaced apart laterally from each other along shaft 652. Thus one tissue holder 654 is closer to the anchor 656 than the other tissue holder 654. Again, the embodiment of FIG. 40 is shown as having two tissue holders 654 but one skilled in the art will understand that more may be employed without departing from the spirit of the invention. Furthermore, a combination of the tissue holders of FIGS. 39 and 40 could be employed. Such a configuration would include multiple tissue holders that are arranged both adjacent to each other as well as longitudinally spaced.

Fasteners 630, 640 and 650 are all shown as having tissue holders 634, 644, and 654, respectively, that are circular once deployed. These tissue holders are preferably formed from a memory-metal, such as nitinol, and are delivered in a relatively straight configuration and curl to a deployed configuration after implantion, as will be discussed in more detail below.

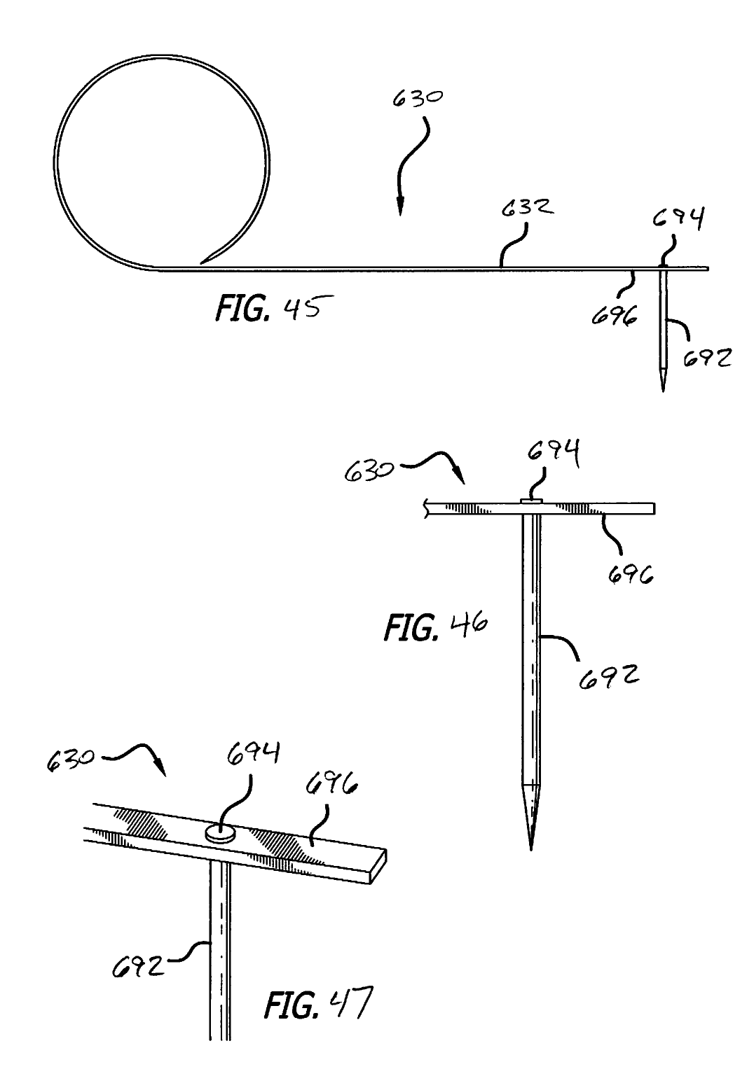

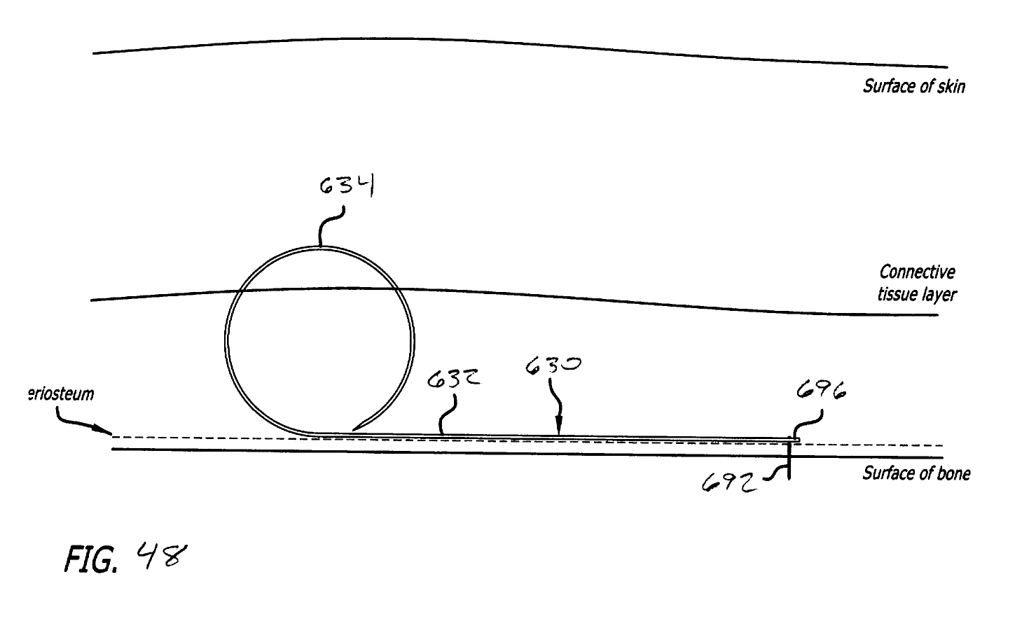

As shown in FIGS. 41 and 42, however, the deployed configuration of the tissue holders is not to be limited to a circular configuration. It may be desired to provide a deeper deployed configuration, such as the fastener 660 shown in FIG. 41, having a complex curve. In FIG. 41, the fastener 660 is partially deployed and the tissue holder 664 extends upward to maximize soft-tissue purchase. In FIG. 42, the fastener 660 has been fully released and has assumed a deployed, fully curved configuration with a low profile.

FIGS. 43 and 44 show elevation and perspective views, respectively, of an embodiment of an anchor 680 of the invention that could be used with any of the embodiments 630, 640, 650, or 660 which are positioned with shafts that are substantially perpendicular with the tissue surface to which the fasteners are attached. FIGS. 43 and 44 demonstrate that an anchor 680 may have multiple anchoring points 682. The embodiment shown in FIGS. 43 and 44 have anchor points that angle toward the other end of the fastener, where the tissue holder is located. This is provided as a non-limiting example.

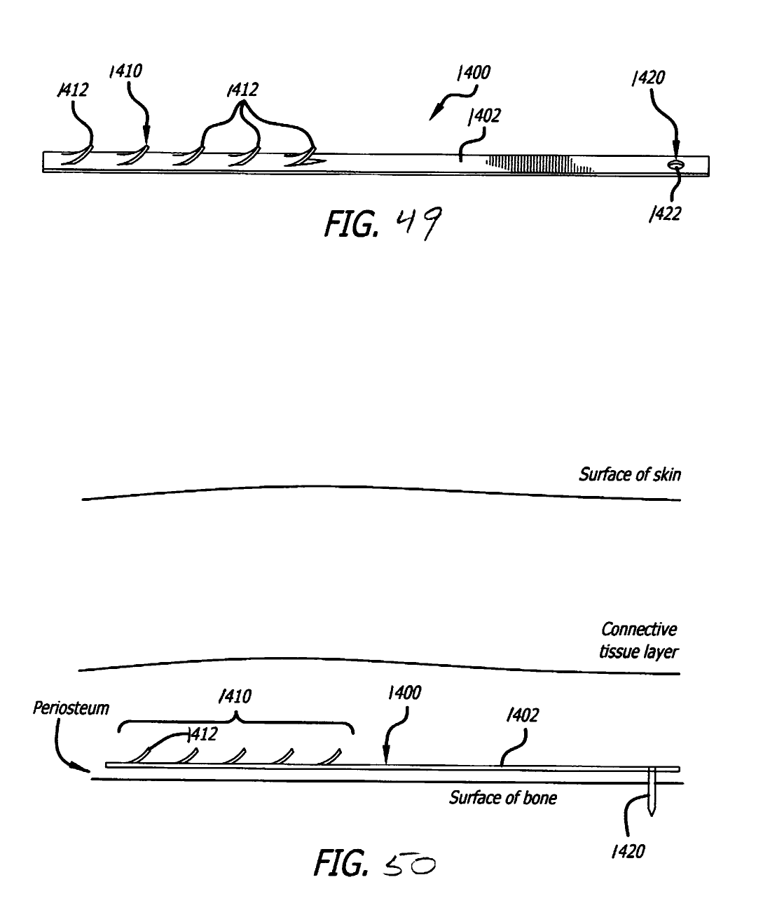

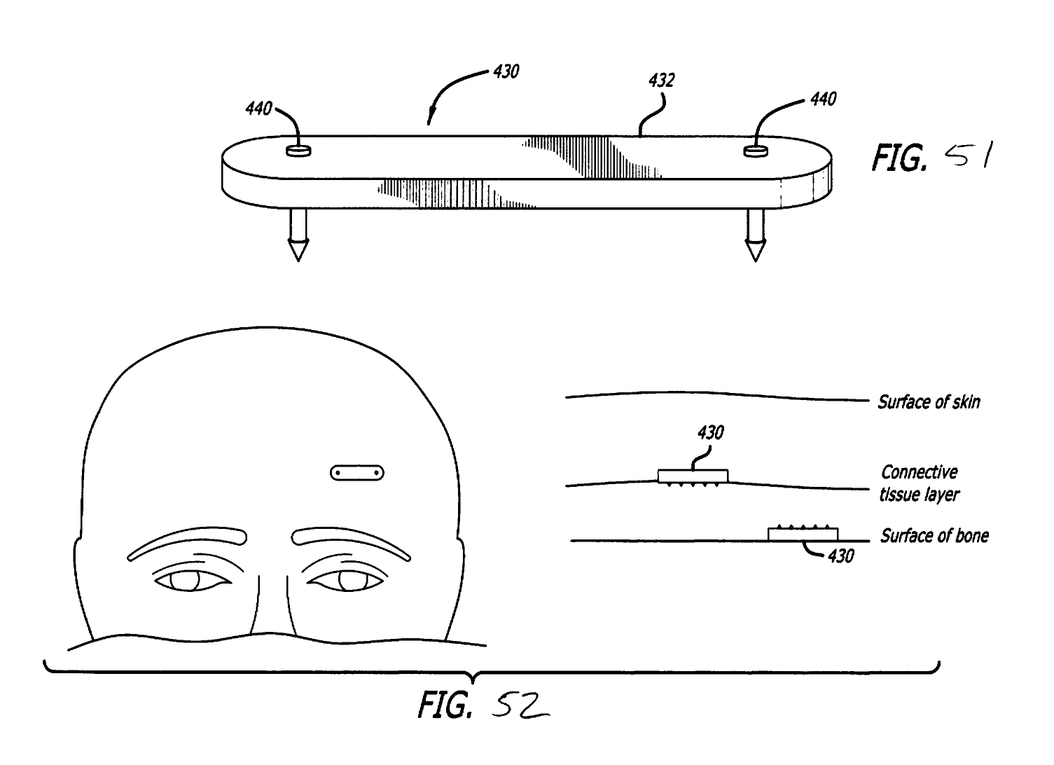

FIGS. 45, 46 and 47 show another embodiment of an anchor 690 that may be used with any of the fastener embodiments 630, 640, 650, or 660. The anchor 690 includes a spike 692 that includes a head 694. The spike passes through the shaft 696 until the head 694 interferes with the shaft 696.

FIG. 48 shows the positioning of a fastener 630 with an anchor 690 relative to the various tissue layers. The periosteum is shown as a dotted line, just above the surface of the bone. It can be seen that the anchor 690 passes through the periosteum into the bone layer. The shaft 632 runs substantially parallel to the bone layer. The tissue holder 634 curls away from the periosteum toward the surface of the skin, but remains below the surface. The tissue holder 634 is sized to engage the connective tissue layer without becoming exposed through the surface of the skin. It is noted that though the anchor 690 appears as a spike, a screw or other similarly-positioned fastener could be used.

Turning now to FIG. 49 there is shown an embodiment of a fastener 1400 having a tissue-holding feature 1410 at one end and an anchor feature 1420 at a second end. Like embodiments 630, 640, 650, and 660, fastener 1400 may be formed of a single, unitary length of material 1402, such as a length of Nitinol ribbon, for example.

The tissue-holding feature 1410 is shown as one or more barbs 1412, which may be cut-out from the length of material 1402. In at least one embodiment, the tissue-holding feature 1410 has a first configuration and a second configuration. In the first configuration, which is assumed in a delivery device, the tissue-holding feature is straight, such that it may be passed through the delivery device. In the second configuration, the barbs 1412 curl outwardly to engage tissue. The second configuration is assumed when the device 1400 is released from the delivery device. The length of material 1402 may be formed of a memory material, such as Nitinol, and the tissue-holding feature 1410 may be biased toward the second configuration.

The anchor feature 1420 of embodiment 1400 includes a hole 1422 for accepting a screw, tack, or other fastener that may be separate or integral with the length of material 1402.

FIG. 50 shows the embodiment 1400 deployed in a patient. In the configuration shown, the fastener 1400 lies on the periosteum and is oriented such that the barbs 1412 extend upward into the connective tissue layer. The anchor feature 1420 passes through the periosteum into the bone layer.

FIG. 51 shows an embodiment 430 of a fastener that includes a length of material 432 having anchor features 440 on each end. The length of material 432 serves as the tissue holding feature and spreads the compressive force placed on the tissue across the surface of the material 432.

FIG. 52 shows the embodiment 430 deployed in a patient and illustrates that the embodiment 430 could be deployed above or below the connective tissue layer.

### Delivery Devices

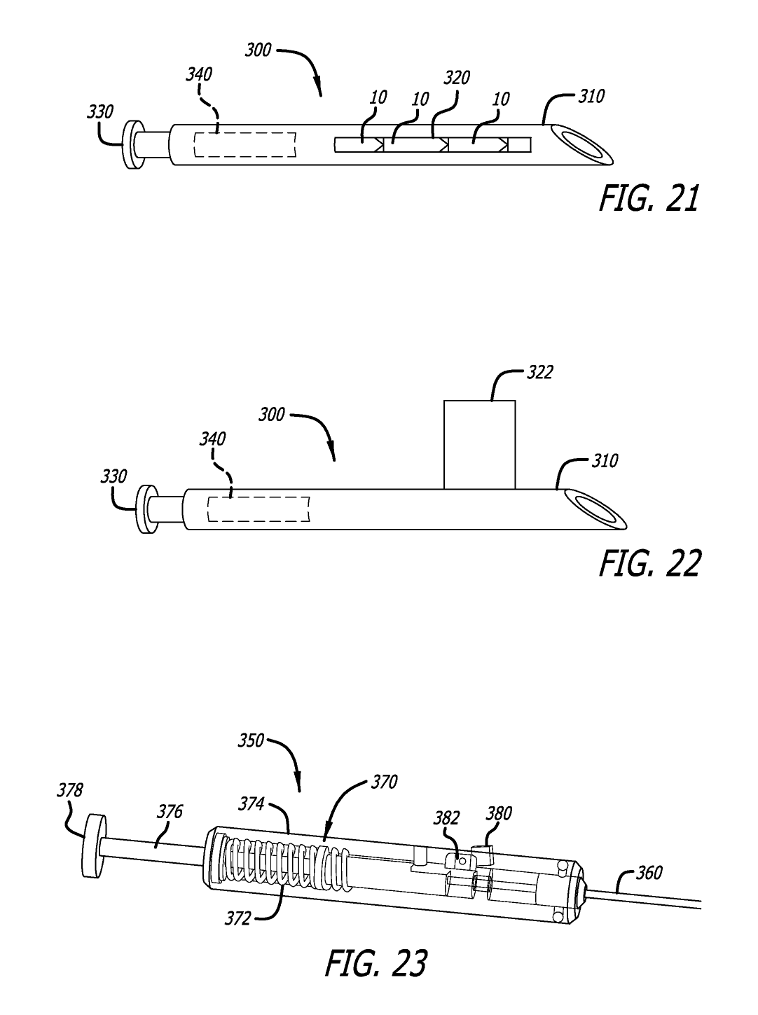

FIGS. 21 and 22 generally show the basic components of a delivery device 300 of the invention. The delivery device includes a hollow needle or barrel 310, a magazine 320 containing one or more anchors 10, a triggering mechanism 330, and a driving mechanism 340.

The barrel 310 may have a sharp tip for making a small incision in the outermost surface of the skin in order to countersink the fastener 10. The barrel also has an interior lumen sized to carry a fastener 10. The magazine 320 may comprise the interior lumen, or may be in the form of a magazine cartridge 322, attachable to the device 300.

The driving mechanism 340 may take on various forms but all are designed to impart an axial force onto the fastener 10. Driving forces that may be useful include pneumatic, spring, electromagnetic, compressed gas such as carbon dioxide, acoustic, ultrasonic, hydraulic impulsion, and the like.

The trigger mechanism 330 is a mechanism used to release or activate the driving force. The release mechanism may be a push button, such as that shown in FIGS. 21 and 22, or may be a finger trigger, foot pedal, bulb, etc.

A more detailed delivery device embodiment 350 is shown in FIG. 23. The delivery device 350 includes a barrel 360, a driving mechanism 370, and a trigger mechanism 380. The driving mechanism 370 includes a spring 372 connected at a distal end to a body 374 and at a proximal end to a piston 376. The trigger mechanism 380 is located on the side of the body and includes a catch 382 that pivots into an interference position with a portion of the piston 376.

In operation, the operator loads a fastener into a lumen of the barrel 360. Alternatively, the fastener is pre-loaded or a magazine is provided with a plurality of fasteners loaded therein. The operator then pulls the proximal end 378 of the piston 376 in a proximal direction. This stretches or compresses the spring 372, depending on how the spring is loaded into the device, providing potential energy for driving the piston 376. The piston 376 is pulled until the catch 382 of the trigger mechanism 380 pivots into an interference position with the distal end of the piston. The delivery device 350 is now loaded and cocked. The operator now gently pulls the skin of a patient or procedure recipient into a desired position, thus shifting the skin layer relative to the bone layer, and holds the skin in the shifted position with his or her finger. The barrel 360 is then pointed at a target site, possibly breaking the skin slightly, and the trigger mechanism 380 is depressed, which pivots the catch 382 out of the interference position, releasing the piston 376. The spring 372 releases its stored energy, and drives the piston axially into a proximal end of a fastener 10, driving the fastener 10 into a bone layer.

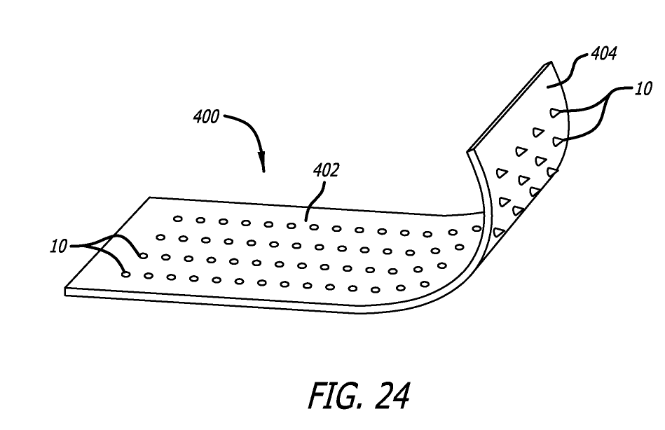

Another embodiment 400 of a delivery device is shown in FIG. 24. Delivery device 400 includes a tape-like substrate having a first side 402 and a second side 404. A plurality of anchors 10 are manufactured into the substrate such that the distal ends of the anchors 10 extend through the second side 404. The second side may include a mild adhesive.

Using the delivery device 400 involves cutting a desired size and shape of the substrate appropriate for the placement of the anchors 10. The targeted skin is appropriately numbed and the substrate 400 is placed on the skin. The adhesive ensures that the substrate will not become accidentally relocated prior to the implantation of the anchors 10. The skin is then shifted to a desired new location and a rubber mallet, or the like, is used to gently tap the anchors 10 into place. Because the anchors 10 are very small, each blow of the mallet contacts several, if not all of the anchors 10, thus requiring few blows. After the anchors 10 are embedded, the substrate 400 is simply peeled away from the patient, leaving the anchors 10 in place.

FIG. 54 shows another embodiment of a delivery device of the invention. The delivery device generally includes a firing gun 800 and a disposable tip 900.

The firing gun 800 is a hand-held, preferably spring-powered gun that accepts the disposable tip 900 at its distal end. Alternatively the firing gun could be disposable and incorporate the distal tip. The firing gun may also be powered by compressed gas, electromagnetic mechanisms or other means.

FIG. 55 shows an exploded view of the firing gun 800 and the disposable tip 900. Beginning near the proximal end of the gun 800, there is a chassis 801 that is a rigid tube made of a hard material such as stainless steel. The chassis 801 has an inner lumen that houses a ram spring 811 and prevents the ram spring 811 from deforming outwardly when compressed. The proximal end of the chassis 801 is closed by a ram plug 803.

The distal end of the ram spring 811 includes ram piece 809 distally protected by a metal ram piece 810, which acts against a firing pin 826. Ram piece 809 may thus be constructed of a lighter material, such as plastic or carbon fiber. The pieces 809 and 810 are contained within the chassis 801 with a chassis cap 802. The firing pin 826 extends through an aperture in the cap 802, so that it can impinge against the disposable tip 900, explained in detail below.

The chassis 801 is contained within a gun-shaped housing 804. The housing 804 includes two clam-shell halves that are held together with screws 816 that mate with threaded inserts 817. The housing 804 slides relative to the chassis 801 by a designated amount, in order to provide a safety feature that requires the device to be pressed against tissue to allow the device to be fired. A housing spring 805 acts between the housing 804 and a lock ring 807 to return the housing 804 to a “safe” position when the device is not being pressed against a surface. The lock ring 807 surrounds the chassis 801 and is fixed relative to the chassis 801 with a set screw 827.

Distal of the lock ring 807, and also surrounding the chassis 801, is a slide ring 812. The slide ring 812 slides relative to the chassis 801 to bring an internal groove formed in the slide ring 812 in and out of alignment with ball bearings 815 of the trigger mechanism. The slide ring 812 is connected to a trigger 814 via two linkage bars 806 and spring pins 808.

The trigger holds the ram 809 in a loaded position in which the ram 809 is compressing the ram spring 811, until fired. This is accomplished with the three ball bearings 815 of the trigger mechanism. The ball bearings 815 ride in holes formed in the chassis 801. The diameter of the ball bearings 815 is greater than the thickness of the wall of the chassis 801. Internally, a groove in the ram 809 provides the additional room needed to house the ball bearings 815. The interference created by the ball bearings between the holes of the chassis 801 and the groove of the ram 809 prevents the ram 809 from being able to be propelled through the chassis 801 by the ram spring 811.

As discussed above, the slide ring 812 slides along the exterior of the chassis 801 and has an internal groove. When the internal groove of the slide ring 812 is aligned with the ball bearing holes of the chassis 801, the ball bearings 815 are forced outwardly by the ram 209 into the space provided by the groove of the slide ring 812. The interference between the ram 809 and the chassis 801 is relieved and the ram 809 is freed and propelled by the ram spring 811.

The trigger 814 affects this chain of events. When pulled, the trigger slides the slide ring 812 until the internal groove of the slide ring 812 is in alignment with the ball bearings 815. A trigger spring 813 returns the trigger to a rest position after it is fired.

As discussed above, the housing 804 has a built in feature whereby unless it is advanced by depressed against a surface, the device will not fire. The trigger 814 and the slide ring 812 are constructed and arranged such that the travel limits of the slide ring 812 prevent the internal groove of the slide ring from aligning with the ball bearings 815 unless the housing 804 is advanced a predescribed distance.

FIG. 56 shows the internal components of the firing mechanism at the moment the device is fired. The housing 804 is advanced by being pressed against a surface, compressing the housing spring 805. The trigger 814 is pulled, against the trigger spring 813, and bringing the internal groove of the slide ring 812 into alignment with the ball bearing holes of the chassis 801.

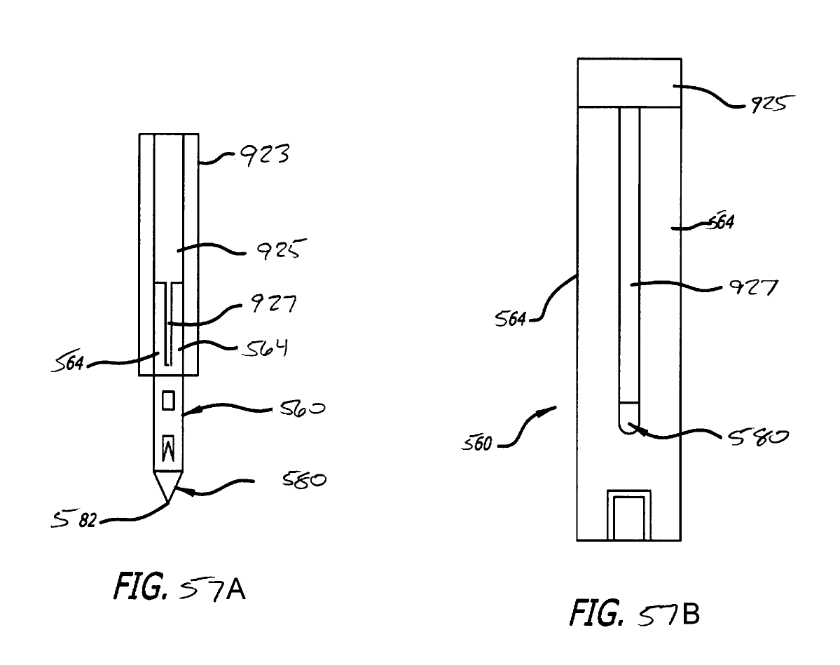

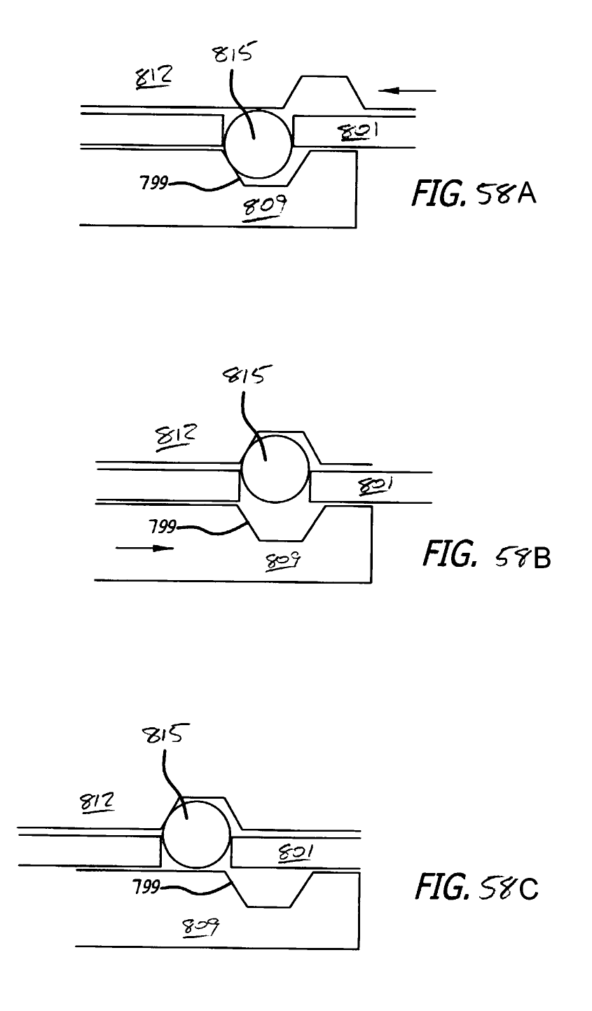

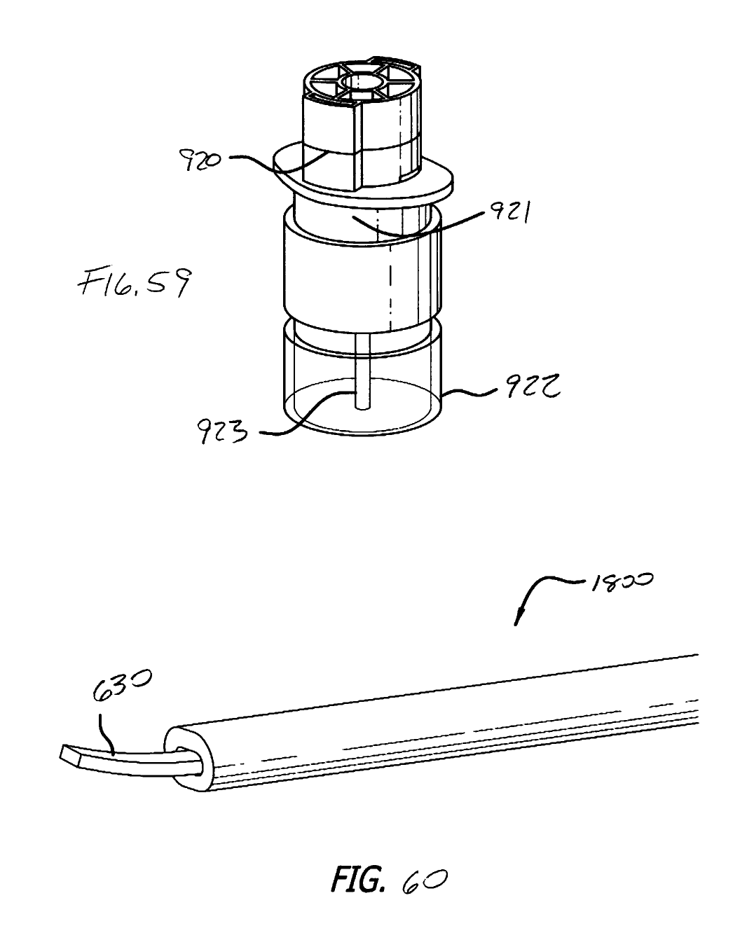

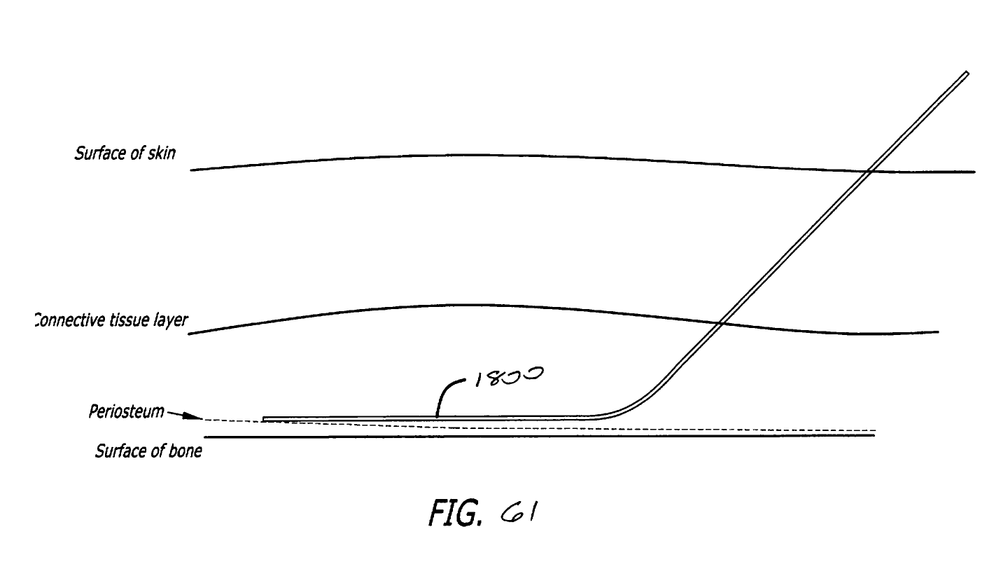

FIGS. 57A and 57B are closeup views of the firing pin 925 as it interacts with a fastener. In this case, the fastener 560 is that of FIG. 27 and has tissue holders 564 in the form of proximal petals. So that the firing pin 925 is acting on the spike 580, and not damaging the petals 564, the firing pin 925 is shaped with a narrower distal end 927 that enters an interior of the fastener 560 and contacts the proximal end of the spike 580.

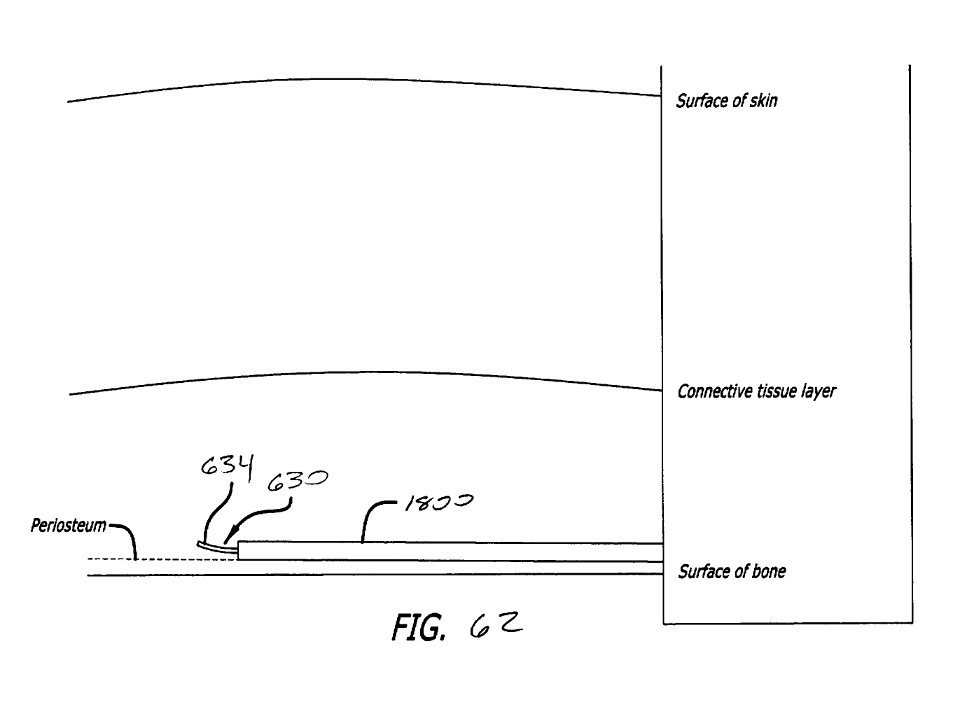

FIGS. 58A-58C show the sequence of events of the firing. In 58A, the ram 809 is unable to be advanced due to the interference between the ram 809 and the chassis 801 created by the ball bearing 815. In 58B, the internal groove of the slide ring 812 is aligned with the ball bearing 815 and the ball bearing 815 is forced into the internal groove of the slide ring 812 by an angled surface 799 of the groove of the ram 809. In 58C, as the interference between the piston 809 and the chassis 801 is no longer being created by the ball bearing 815, the ram 809 is freed and propelled forward.

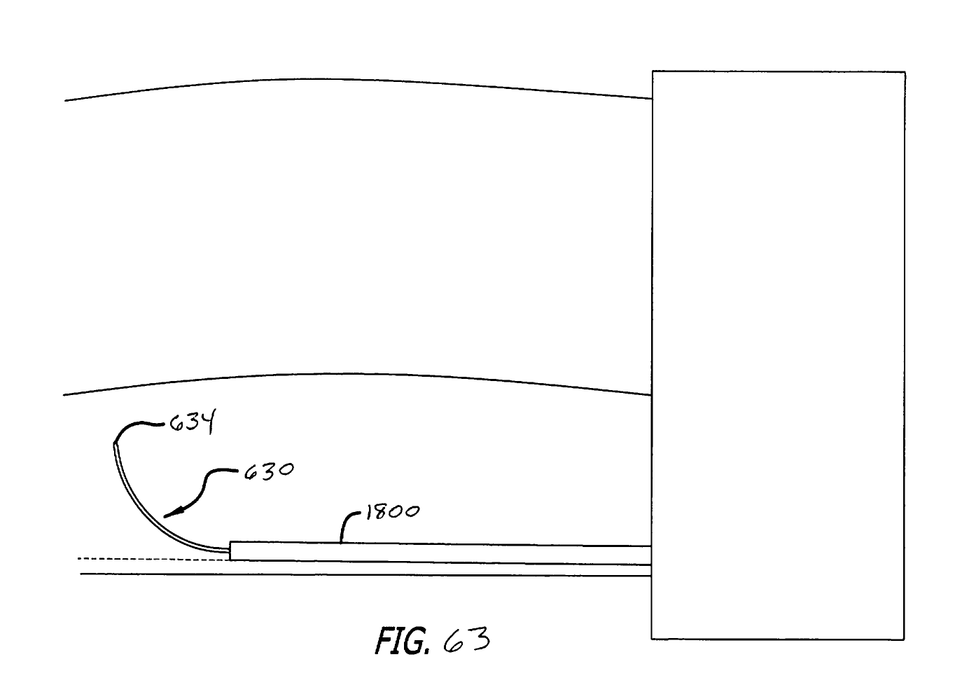

Referring again to FIG. 55, and also to FIG. 59, the disposable tip 900 includes, beginning from a proximal end, an outer firing pin rod 928, an inner firing pin rod 925, a proximal tip component 920, hypotube 924, hypotube 923, a tip spring 921, and a distal tip component 922. Loaded into the distal end of the distal hypotube (or cannula) 923 is the implant and the spike (shown as components 919 and 918 but could be any of the implants and spikes shown and described herein).

The proximal tip component 920 is a plastic piece that is used to attach the tip 900 to the firing gun 800. The proximal tip component 920 includes a mating component that mates with a corresponding mating component at the distal end of the housing 804.

As best seen in FIG. 59, the distal tip component 922 is slidably mated via grooves with the proximal tip component and is biased toward a separated state therewith by a tip spring 921. The tip spring 921 is thus positioned as a shock absorber and allows the physician to press the distal end of the distal component 922 against the patient without undue discomfort. In this regard, the distal component 922 acts as a stabilizer against the skin of the patient. The distal component 922 also acts as an alignment device to aid in orientating the cannula 923 perpendicularly to the surface of the implantation site.

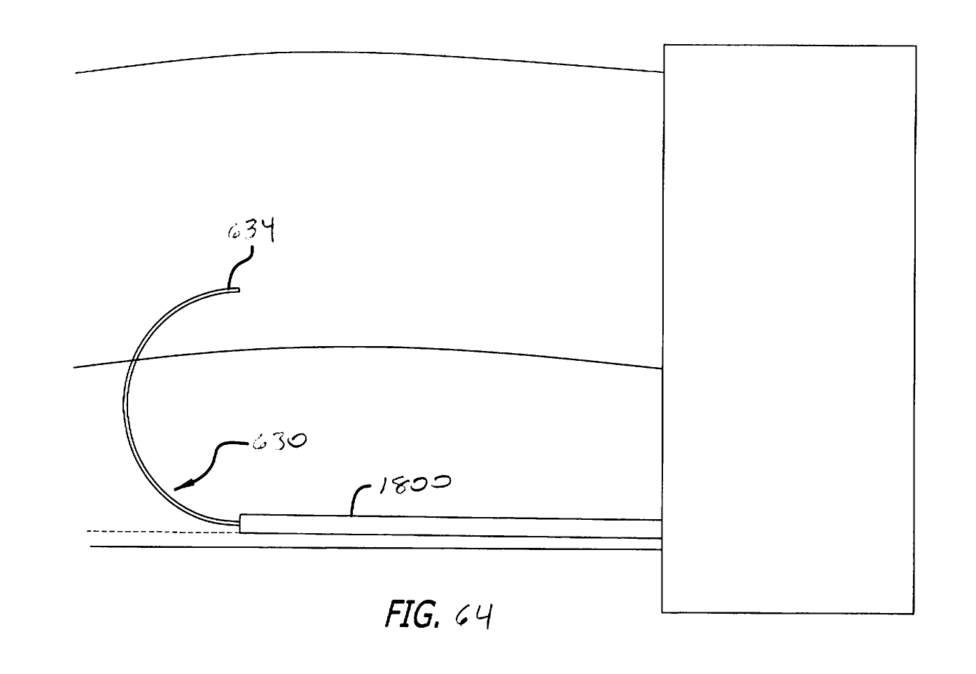

The fastener embodiments 630, 640, 650, and 660 may be used with a different delivery device 1800. Referring to FIG. 60, there is shown a delivery device 1800 that is in the form of a low-profile catheter shaped to slideably house the fastener. Fastener 630 is used as an example in FIG. 60.

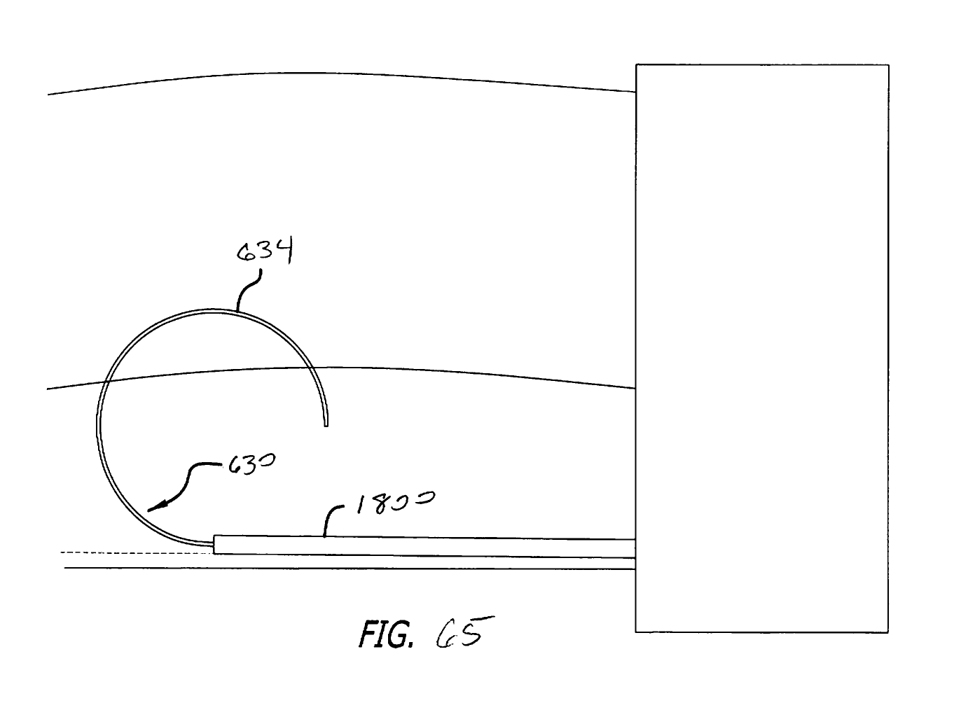

FIG. 61 shows the first in a series of delivery steps utilizing the delivery device 1800. As can be seen, the delivery device 1800 is routed through an incision along the periosteum until a target location is reached. At this point, as best seen in FIG. 62, the fastener 630 is ejected from the distal end of the delivery device 1800. The distal end of the fastener 630 is the tissue holder 634.

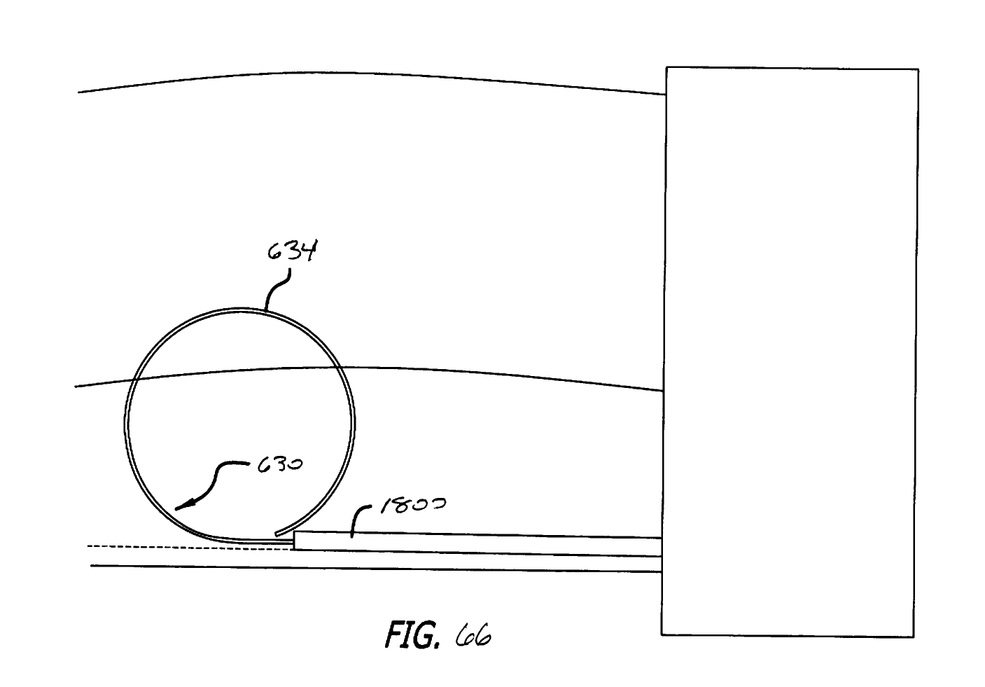

FIGS. 63 and 64 show the tissue holder 634 curling upwardly toward the tissue surface as the fastener 630 continues to be ejected from the delivery device 1800. As the tissue holder 634 curls, the holder 634 engages and holds onto tissue.

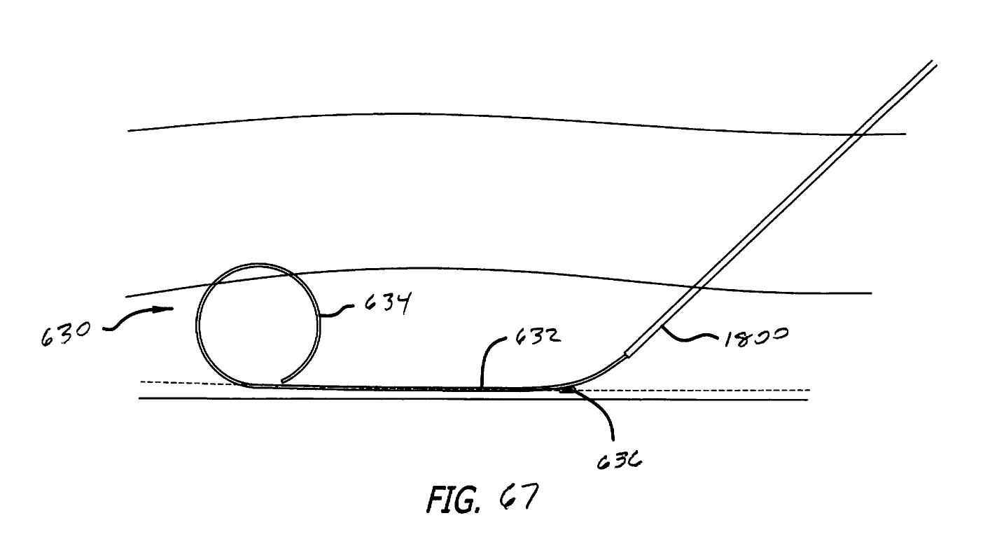

FIGS. 65 and 66 show the tissue holder 634 curling back toward the delivery device 1800. One skilled in the art will realize that different degrees of curling could be utilized and may vary depending on the location and type of tissue being engaged.

In FIG. 67, the tissue holder 634 has been fully deployed and the delivery device 1800 has been retracted from the target site, fully exposing the shaft 632 and the anchor 636. Due to the elastic forces the surface skin layers place on the tissue holder 634, the anchor 636 naturally engages the periosteum. However, downward force may be placed on the anchor 636 to enhance engagement of the periosteum.

Though the delivery device 1800 is shown as deploying a fastener 630 with the tissue holder 634 emerging first, it is contemplated that the fastener 630, 640, 650, or 660 could be loaded into the delivery device 1800 in a reverse order such that the anchor 636, etc., emerges first and engages the periosteum immediately while the tissue holder 634, etc., emerges last.

### Operation Dynamics

Through computation and experimentation, it has been determined that given the relatively hard bone of the human skull, a sharp, solid pin is preferred to initiate penetration. However, fasteners may also be configured as a sharpened hypotube without the central pin. For any given implant configuration, including factors such as fastener diameter, length, sharpness, tissue characteristics, and the like, there is minimum energy threshold that is required to drive the fastener effectively into the bone to the desired depth. Furthermore, various types of bone vary in their thickness and hardness, which effects the energy necessary for implantation, as well as the strength and features needed in the implant itself.

The embodiments of the driving devices described herein use a spring. The potential energy stored in the ram spring is converted quickly to kinetic energy when the spring is released. That kinetic energy is transferred, in turn, to the fastener. Though spring energy is used in the embodiments described herein, one skilled in the art will understand that pneumatic, ultrasound, electric, or a variety of other energy sources, could be used to achieve the results of the invention.

It has been determined that if one exceeds the minimum energy level for implantation, the depth that the fastener is driven into the skull can be controlled by the length of travel of the piston/firing pin. Because the fastener has very little mass, and therefore very little momentum, the fastener will not continue to travel deeper into the tissue once the energy source is stopped or otherwise isolated from the fastener. Other means of controlling fastener depth are possible, including controlling the total energy applied to the fastener, or the depth of a pre-drilled hole.

Failure to achieve the minimum energy level typically results in a deformation of the fastener as it is being driven into the skull, or at a minimum results in an fastener that is delivered to a sub-optimal depth. It is thought that a failure to achieve the minimum energy level results in a reduced fastener velocity, which gives the fastener additional time to deform upon entry into the skull. At normal speeds, during a perfectly inelastic collision, an object struck by a projectile will deform, and this deformation will absorb most or all of the force of the collision. Viewed from a conservation of energy perspective, the kinetic energy of the projectile is changed into heat and sound energy, as a result of the deformations and vibrations induced in the struck object. However, these deformations and vibrations cannot occur instantaneously. A high-velocity collision (an impact) does not provide sufficient time for these deformations and vibrations to occur. Thus, the struck material behaves as if it were more brittle than it would otherwise be, and the majority of the applied force goes into fracturing the material.

This minimum energy is thus factored into the selection of the spring strength, the travel distance and mass of the metal ram 810 and ram 809. The spring must be strong enough, and the travel of the ram 810 and 809 must be long enough, such that speed of the ram 810 and 809 achieves a minimum velocity prior to impacting the firing pin 826. Additionally the mass of 809 and 810 must be such that the kinetic energy of the spring is sufficient to accelerate 809 and 810 to a critical velocity. This mass also plays a role in the amount of momentum that is transferred to the firing pin. Alternatively, the ram 810 and 809 and the firing pin 826 could comprise a single piece, in which case the pin 826 would be propelled to a minimum velocity by the ram spring 811 prior to impacting the fastener assembly.

Assuming the minimum fastener velocity is achieved, the depth of the fastener can be controlled in a variety of ways. As stated above, one way to control depth is to limit the travel of the firing pin. As the fastener has very little mass, the fastener will not continue to travel beyond the travel of the firing pin. It is also possible to control fastener depth or pin travel by precisely controlling the energy delivered to the fastener or pin.

It is also possible to control firing pin travel using other methods, some of which allow a lighter ram spring to be used. For example, predrilling a hole for the fastener allows the fastener to be driven using a lighter driving force. Both the depth of the pre-drilled hole, as well as the diameter of the pre-drilled hole relative to the diameter of the fastener can be varied to control the resulting fastener depth.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for relocating a skin layer relative to an adjacent bone layer comprising:
shifting a skin layer relative to an adjacent bone layer from an original position to a shifted position without cutting muscle or connective tissue associated with said skin layer;
holding said skin layer in said shifted position while inserting a fastener through said skin layer into said bone layer to prevent said skin layer from reassuming said original position.

2. The method of claim 1 further comprising inserting one or more additional fasteners through said skin layer into said bone layer.

3. The method of claim 1 wherein inserting a fastener through said skin layer into said bone layer comprises inserting a fastener through said skin layer into said bone layer such that an outer-most feature of said fastener is located just below an outer surface of said skin layer.

4. The method of claim 1 wherein inserting a fastener through said skin layer comprises ballistically inserting a fastener through said skin layer.

5. The method of claim 4 wherein ballistically inserting a fastener through said skin layer comprises driving said fastener into said bone layer using a spring-loaded driving mechanism.

6. The method of claim 4 wherein ballistically inserting a fastener through said skin layer comprises driving said fastener into said bone layer using a compressed-gas driving mechanism.

7. The method of claim 1 wherein inserting a fastener through said skin layer comprises inserting a fastener having an open hollow tip.

8. The method of claim 1 wherein inserting a fastener through said skin layer comprises inserting a fastener having an anchoring feature proximate a distal end thereof for preventing said fastener from becoming dislodged from said bone layer.

9. A fastener for use in anchoring a skin layer in a shifted position to a bone layer thereby overcoming lateral and axial forces imparted by said skin layer on said fastener comprising at least one shaft having a distal end and a proximal end, said at least one shaft including:
an anchoring feature proximate said distal end; and,
a tissue-holding feature proximate said proximal end.

10. The fastener of claim 9 wherein said at least one shaft comprises a plurality of shafts and said tissue holding feature comprises at least one bridge connecting one of said plurality of shafts to another one of said plurality of shafts.

11. The fastener of claim 9 wherein said at least one shaft comprises an hollow portion that is open at said distal end.

12. The fastener of claim 9 wherein said anchoring feature comprises at least one barb.

13. The fastener of claim 9 wherein said fastener comprises a bioabsorbable material.

14. The fastener of claim 9 wherein said tissue holding feature comprises at least one protrusion that extends radially from a longitudinal axis of said shaft when said fastener is driven into said bone layer.

15. The fastener of claim 9 further comprising a stop that limits a depth that said fastener can be driven into said bone layer.

16. The fastener of claim 15 wherein said shaft comprises a hollow portion that is open at said distal end and said stop comprises a closed proximal end of said hollow portion.

17. A device for use in anchoring a skin layer in a shifted position relative to a bone layer thereby overcoming lateral and axial forces imparted by said skin layer on said fastener comprising at least one shaft having a distal end and a proximal end comprising:
a delivery mechanism; and,
at least one fastener;
said delivery mechanism including:
a barrel;
a driving mechanism that drives said at least one mechanism out of a distal end of said barrel;
said at least one fastener including at least one shaft having:
an anchoring feature proximate said distal end; and,
a tissue holding feature proximate said proximal end.

18. The device of claim 17 wherein said delivery mechanism further includes a cartridge containing a plurality of said fasteners.

19. The device of claim 17 wherein said driving mechanism comprises:
a spring;
a piston driven by said spring and having a distal end that transfers an axial force released by said spring onto a proximal end of said fastener;
a triggering mechanism for releasing energy stored in said spring.

20. The device of claim 17 wherein said driving mechanism comprises:
a capsule containing pressurized gas;
a piston driven by said pressurized gas and having a distal end that transfers an axial force onto a proximal end of said fastener;
a triggering mechanism for releasing said axial force stored in said spring.
